(19) United States
(12) Patent Application Publication
(10) Pub. No.: US 2023/0151324 A1
(43) Pub. Date: May 18, 2023

(54) PRESERVATION SYSTEM FOR STABILIZING SPORE-FORMING MICROBIALS

(71) Applicant: Phibro Animal Health Corporation, Teaneck, NJ (US)

(72) Inventor: William W. Waugh, Sarasota, FL (US)

(73) Assignee: Phibro Animal Health Corporation, Teaneck, NJ (US)

(21) Appl. No.: 17/983,982

(22) Filed: Nov. 9, 2022

Related U.S. Application Data
(60) Provisional application No. 63/279,903, filed on Nov. 16, 2021.

(51) Int. Cl. C12N 1/20; C12N 1/04

(52) U.S. Cl. CPC C12N 1/20; C12N 1/04

(57) ABSTRACT

Disclosed embodiments concern concentrated compositions comprising a microbial suspension and a preservative system, such as a paraben preservative, an isothiazolinone preservative, and a quaternary ammonium preservative. Microbes used to form the compositions generally are spore-forming microbes, such as bacteria or fungi, with representative embodiments concerning compositions comprising Bacillus. Concentrated compositions may be formulated by combining a Prep A composition comprising a microbial suspension with a Prep B composition comprising a preservative system. Preserved concentrated microbial compositions may be formulated at any of various concentrations relative to diluted, ready-to-use compositions, such as 40×, 50×, 80×, 100× and 250× compositions. Diluted microbial suspension compositions can be used as a cleaning product, an odor control aid, as a janitorial product, as an agricultural product, as a product to treat wastewater, as an environmental product, or as a processing aid.

### CROSS REFERENCE TO RELATED APPLICATION

This application claims the benefit of the earlier filing date of U.S. provisional patent application No. 63/279,903, filed Nov. 16, 2021, which is incorporated herein by reference in its entirety.

### FIELD

The present application concerns a preservative system for compositions comprising microbial cells or spores, such as bacterial cells and spores, a microbial composition comprising the preservative system wherein the microbial composition may be concentrated by at least 10 times to at least 500, such as 40 times to at least 250 times, and more typically 80 times to 250 times, the concentration intended for an end-use application, and methods for making and using such compositions.

### BACKGROUND

A number of compositions comprising bacterial cultures are commercially important. Currently, the bacterial cultures are formulated as very dilute solutions in order to form a stable composition for storage and shipping. There are a number of disadvantages associated with these dilute solutions. For example, the expenses associated with shipping and storing large quantities of dilute bacterial cultures are substantially increased because a substantial portion of the weight associated with these compositions is water. Currently, however, as such bacterial compositions are concentrated, the likelihood for contamination by, for example, another bacteria, fungus and/or yeast substantially increases; therefore, the viability and stability of such concentrated bacterial compositions substantially decreases. The assignee of the present application has, for example, found it difficult to formulate stable, preserved bacterial compositions at concentrations much higher than about 5 times a concentration intended for an end-use application

As a result, a need exists for concentrated bacterial cultures that reduce shipping costs and increase culture stability.

### SUMMARY

The present invention address these and other problems associated with microbial suspension formulations. Disclosed embodiments of a concentrated microbial composition generally comprise a microbial suspension and a preservative system, typically comprising plural different preservatives. The concentrated microbial compositions typically are diluted for an end-use application, and therefore the concentrated microbial compositions may be concentrated by at least 10 times to at least 500, such as 40 times to at least 250 times, and more typically 80 times to 250 times, the concentration intended for an end-use application. Microbes used to form the compositions generally are spore-forming microbes, such as bacteria or fungi. Certain disclosed representative concentrated microbial compositions comprise Bacillus, such as Bacillus coagulans, Bacillus pumilus, Bacillus thuringiensis, Bacillus inaquosorum, Bacillus mojavensis, Bacillus megaterium, Bacillus globigii, Bacillus subtilis, Bacillus simplex, Bacillus licheniformis and/or Bacillus amyloliquefaciens. Concentrated microbial compositions may include a solvent, such as a humectant solvent, such as an alcohol, a diol, a triol, an alkylene glycol, a glycerol or combinations thereof, with propylene glycol, glycerol, or PEG 400 being used for certain representative embodiments. The solvent may be used in suitable amounts, such as greater than 0% to 99% by weight solvent, more typically 10% to 60% by weight solvent, and even more typically 30% to 50% solvent. A person of ordinary skill in the art will understand that concentrated microbial compositions may include additional components, as may be beneficial for various end-use applications, such as at least one additional component selected from a buffering system, a non-ionic surfactant, a surfactant, such as Tween 80, odor mitigation components, deodorizers, fragrances, opacifiers, enzymes, such as a cellulase or lipase, proteins, gelling agents, additional preservatives, sanitizing agents, emulsifying agents, API's, oils such as triglycerides, antioxidants, oxidizers, petroleum products, biocides, solvents, and any and all combinations thereof.

Certain disclosed embodiments of concentrated microbial compositions include at least 3 different preservatives, such as a paraben preservative, an isothiazolinone preservative, and a quaternary ammonium preservative. Suitable quaternary ammonium preservatives may have a formula R¹R²R³R⁴N⁺X⁻, where R¹-R⁴ are the same or different, and are independently selected from H, C₁-C₂₀ alkyl, and aryl. Representative quaternary ammonium preservatives include benzalkonium chloride, such as C₈₋₁₈ n-alkyl dimethyl benzyl ammonium chloride, cetyl pyridinium chloride, octylphenoxyethoxyethyl dimethyl benzyl ammonium chloride, and combinations thereof. Examples of suitable paraben preservatives include methylparaben and propylparaben, and exemplary isothiazolinone preservatives may be selected from 1,2-benzoisothiazolin-3-one (BIT), methylisothiazolinone (MIT), methylchloroisothiazolinone (CMIT), and combinations thereof. Benzalkonium chloride, 1,2-benzoisothiazolin-3-one, and propylparaben were used to prepare particular disclosed compositions.

Concentrated compositions may be formulated using a Prep A composition comprising a microbial suspension and a Prep B composition comprising a preservative system. Prep A compositions are combined with Prep B compositions to form preserved concentrated microbial compositions according to the present disclosure. A particular disclosed Prep A microbial composition comprised at least one Bacillus species, a buffer system, such as monopotassium phosphate, and dipotassium phosphate, a non-aqueous solvent, typically a humectant such as propylene glycol, and water. As an example, a particular Prep B composition for a 250× formulation comprised 28% to 30% Proxel GXL comprising 20% benzisothiazolinone, 14% to 15% Stepenquat 65 NF comprising 50% n-alkyldimethylbenzylammonium chloride, 14% to 15% propylparaben, and 42% to 43% propylene glycol.

The preserved concentrated microbial compositions may be formulated at any of various concentrations relative to diluted, ready-to-use compositions, such as 40×, 50×, 80×, 100× and 250× compositions. The amount to which preserved concentrated microbial compositions are diluted to form ready-to-use formulations can best be determined by considering particular end-use applications. But, as a general rule, the amount of dilution can be at least substantially equal to the degree to which compositions according to the present invention are concentrated. For example, a 500× concentrate may be diluted up to 1/500× for an end-use application; a 250× concentrate may be diluted up to 1/250× for an end-use application; etc. Dilutions also can be determined as a percentage of the concentrate concentration, and can vary from greater than 0% to 100% dilution, but more typically are equal to ±30%. For example, a 250× concentrate may be diluted within the dilution range of from 1/175 to 1/325, which is equal to ±30% relative to the initial concentrate concentration.

A particular disclosed 100× concentrate composition comprises, consists of or consists essentially of 5 to 7×10⁹ CFU/mL bacterial Cells and/or spores, 36% propylene glycol, 2% propylparaben, 4% benzoisothiazolone, 2% n-alkyl dimethyl benzyl ammonium chloride, 1% dipotassium phosphate, 0.6% monopotassium phosphate, optionally 0.1% Tween 80, and water to volume. A particular disclosed 250× concentrate composition comprises, consists of or consists essentially of 1 to 2×10¹⁰ CFU/mL bacterial Cells and/or spores, 41% propylene glycol, 5% propylparaben, 2% benzoisothiazolone, 2.5% n-alkyl dimethyl benzyl ammonium chloride, 2.5% dipotassium phosphate, 1.5% monopotassium phosphate, optionally 0.1% Tween 80, and water to volume.

A method for making concentrated, stabilized and preserved microbial suspension compositions or combinations also is disclosed. The method may comprise forming a microbial Prep A suspension, forming a Prep B preservative system, and combining the Prep A and Prep B formulations to form concentrated, stabilized and preserved microbial suspensions.

Embodiments of a method for using concentrated, stabilized and preserved microbial suspension compositions also are disclosed. One embodiment comprises providing or obtaining disclosed embodiments of a concentrated, stabilized and preserved microbial suspension composition, diluting the concentrated, stabilized and preserved microbial suspension composition with water, an alkylene glycol or a combination thereof to form a diluted microbial suspension composition, and then using the diluted microbial suspension composition. The diluted microbial suspension composition can be used for any of various end-use applications, such as a cleaning product, an odor control aid, a composition useful for draining clogged lines, as a janitorial product, as an agricultural product, as a product to treat waste water, as an environmental product, or as a processing aid.

The foregoing and other objects, features, and advantages of the invention will become more apparent from the following detailed description, which proceeds with reference to the accompanying figures.

### BRIEF DESCRIPTION OF THE DRAWINGS

FIG. 1 is a challenge plate illustrating results obtained by diluting a concentrated, stabilized and preserved microbial suspension composition according to the present invention and challenging the diluted composition with a gram-negative bacterial challenge relative to a control at time T₀.

FIG. 2 is a challenge plate illustrating results obtained by diluting a concentrated, stabilized and preserved microbial suspension composition according to the present invention and challenging the diluted composition with a gram-negative bacterial challenge relative to a control at time T₀.

FIG. 3 is a challenge plate illustrating results obtained by diluting a concentrated, stabilized and preserved microbial suspension composition according to the present invention and challenging the diluted composition with a gram-negative bacterial challenge relative to a control at time T₁.

FIG. 4 is a challenge plate illustrating results obtained by diluting a concentrated, stabilized and preserved microbial suspension composition according to the present invention and challenging the diluted composition with a gram-negative bacterial challenge relative to a control at time T₁.

FIG. 5 is a challenge plate illustrating results obtained by diluting a concentrated, stabilized and preserved microbial suspension composition according to the present invention and challenging the diluted composition with a yeast challenge relative to a control at time T₃.

FIG. 6 is a challenge plate illustrating results obtained by diluting a concentrated, stabilized and preserved microbial suspension composition according to the present invention and challenging the diluted composition with a yeast relative to a control at time T₃.

FIG. 7 is a challenge plate illustrating results obtained by diluting a concentrated, stabilized and preserved microbial suspension composition according to the present invention and challenging the diluted composition with both yeast and mold relative to a control at time T₀.

FIG. 8 is a challenge plate illustrating results obtained by diluting a concentrated, stabilized and preserved microbial suspension composition according to the present invention and challenging the diluted composition with both yeast and mold relative to a control at time T₀.

FIG. 9 is a challenge plate illustrating results obtained by diluting a concentrated, stabilized and preserved microbial suspension composition according to the present invention and challenging the diluted composition with a gram-negative bacterial challenge relative to a control at time T₀.

FIG. 10 is a challenge plate illustrating results obtained by diluting a concentrated, stabilized and preserved microbial suspension composition according to the present invention and challenging the diluted composition with a gram-negative bacterial challenge relative to a control at time T₀.

FIG. 11 is a challenge plate illustrating results obtained by diluting a concentrated, stabilized and preserved microbial suspension composition according to the present invention and challenging the diluted composition with both yeast and mold relative to a control at time T₀.

FIG. 12 is a challenge plate illustrating results obtained by diluting a concentrated, stabilized and preserved microbial suspension composition according to the present invention and challenging the diluted composition with both yeast and mold relative to a control at time T₀.

FIG. 13 is a challenge plate illustrating results obtained by diluting concentrated, stabilized and preserved microbial suspension composition according to the present invention and challenging the diluted compositions with a gram-negative bacterial challenge relative to a control at time T₀.

FIG. 14 is a challenge plate illustrating results obtained by diluting a concentrated, stabilized and preserved microbial suspension composition according to the present invention and challenging the diluted composition with a gram-negative bacterial challenge relative to a control at time T₀.

FIG. 15 is a challenge plate illustrating results obtained by diluting a concentrated, stabilized and preserved microbial suspension composition [Lead Bacillus 1/250× T(stock+15 and 385)] according to the present invention and challenging the diluted composition with a gram-negative bacterial challenge relative to a control at time T₀. FIGS. 15, 16, 19 and 20 do illustrate positive response for bacterial presence at T+0; however, loss at T+0 is not expected because insufficient time has elapsed to provide cidal activity. In an antiseptic test, immediate die-off would be desired, but for microbial preservation the goal is to prevent or reduce growth over time. Cidal activity is evidence after T+1 in most instances, which is a better indicator of effectiveness, as it shows inability to grow or survive in the presence of preservatives. +15 day and +385 day samples were ready to use ready-to-use RTU compositions that were prepared from the same stock; the former was prepared 15 days after the stock was produced, and the latter was 385 days after stock production. The +15 day and +385 day samples establish that disclosed systems according to the present invention still provide preservation in an old concentrate.

FIG. 16 is a challenge plate illustrating results obtained by diluting a concentrated, stabilized and preserved microbial suspension composition [Lead Bacillus 1/250× T(stock+15 and 385)] according to the present invention and challenging the diluted composition with a gram-negative bacterial challenge relative to a control at time T₀.

FIG. 17 is a challenge plate illustrating results obtained by diluting a concentrated, stabilized and preserved microbial suspension composition [Lead Bacillus 1/250× T(stock+15 and 385)] according to the present invention and challenging the diluted composition with a gram-negative bacterial challenge relative to a control at time T₂=2 days subsequent to formulation.

FIG. 18 is a challenge plate illustrating results obtained by diluting a concentrated, stabilized and preserved microbial suspension composition [Lead Bacillus 1/250× T(stock+15 and 385)] according to the present invention and challenging the diluted composition with a gram-negative bacterial challenge relative to a control at time T₂=2 days subsequent to formulation.

FIG. 19 is a challenge plate illustrating results obtained by diluting a concentrated, stabilized and preserved microbial suspension composition [bacillus 1/250× T(stock+15 and 385)] according to the present invention and challenging a diluted composition with yeast relative to a control at time T₀.

FIG. 20 is a challenge plate illustrating results obtained by diluting a concentrated, stabilized and preserved microbial suspension composition [bacillus 1/250× T(stock+15 and 385)] according to the present invention and challenging a diluted composition with yeast relative to a control at time T₀.

FIG. 21 is a challenge plate illustrating results obtained by diluting a concentrated, stabilized and preserved microbial suspension composition [Lead Bacillus 1/250× T(stock+15 and 385)] according to the present invention and challenging the diluted composition with a yeast challenge relative to a control at time T₂=2 days subsequent to formulation.

FIG. 22 is a challenge plate illustrating results obtained by diluting a concentrated, stabilized and preserved microbial suspension composition [Lead Bacillus 1/250× T(stock+15 and 385)] according to the present invention and challenging the diluted composition with a yeast challenge relative to a control at time T₂=2 days subsequent to formulation.

### DETAILED DESCRIPTION

### I. Terms

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing embodiments from discussed prior art, the embodiment numbers are not approximates unless the word “about” is recited.

Although there are alternatives for various components, parameters, operating conditions, etc. set forth herein, that does not mean that those alternatives are necessarily equivalent and/or perform equally well. Nor does it mean that the alternatives are listed in a preferred order unless stated otherwise.

Definitions of common terms in chemistry may be found in Richard J. Lewis, Sr. (ed.), Hawley's Condensed Chemical Dictionary, published by John Wiley & Sons, Inc., 2016 (ISBN 978-1-118-13515-0). Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar references.

In order to facilitate review of the various embodiments of the disclosure, the following explanations of specific terms are provided:

Alcohol: An organic compound including at least one hydroxyl group. Alcohols may be monohydric (including one —OH group), dihydric (including two —OH groups; diols, such as glycols), trihydric (including three —OH; triols, such as glycerol) groups, or polyhydric (including three or more —OH groups; polyols). The organic portion of the alcohol may be aliphatic, cycloaliphatic (alicyclic), heteroaliphatic, cycloheteroaliphatic (heterocyclic), polycyclic, aryl, or heteroaryl, and may be substituted or unsubstituted.

Ammonium: A cation having a formula [N(H)(R′)₃]⁺ where each R′ independently is H, alkyl, aryl, etc.

Bacteria: Single-cell, prokaryotic (i.e., without a nucleus) organisms.

Lower: Refers to organic compounds having 10 or fewer carbon atoms (C₁₋₁₀) in a chain, including all branched and stereochemical variations, particularly including methyl, ethyl, propyl, butyl, pentyl, hexyl, heptyl, octyl, nonyl, and decyl.

Solution: A homogeneous mixture composed of two or more substances. A solute (minor component) is dissolved in a solvent (major component). A plurality of solutes and/or a plurality of solvents may be present in the solution.

Substituent: An atom or group of atoms that replaces another atom in a molecule as the result of a reaction. The term “substituent” typically refers to an atom or group of atoms that replaces a hydrogen atom, or two hydrogen atoms if the substituent is attached via a double bond, on a parent hydrocarbon chain or ring. The term “substituent” may also cover groups of atoms having multiple points of attachment to the molecule, e.g., the substituent replaces two or more hydrogen atoms on a parent hydrocarbon chain or ring. In such instances, the substituent, unless otherwise specified, may be attached in any spatial orientation to the parent hydrocarbon chain or ring. Exemplary substituents include, for instance, alkyl, alkenyl, alkynyl, alkoxy, alkylamino, alkylthio, acyl, aldehyde, amido, amino, aminoalkyl, aryl, arylalkyl, arylamino, carbonate, carboxyl, cyano, cycloalkyl, dialkylamino, halo, haloaliphatic (e.g., haloalkyl), haloalkoxy, heteroaliphatic, heteroaryl, heterocycloaliphatic, hydroxyl, oxo, sulfonamide, sulfhydryl, thio, and thioalkoxy groups.

Substituted: A fundamental compound, such as an aryl or aliphatic compound, or a radical thereof, having coupled thereto one or more substituents, each substituent typically replacing a hydrogen atom on the fundamental compound. A person of ordinary skill in the art will recognize that compounds disclosed herein may be described with reference to particular structures and substituents coupled to such structures, and that such structures and/or substituents also can be further substituted, unless expressly stated otherwise or context dictates otherwise. Solely by way of example and without limitation, a substituted aryl compound may have an aliphatic group coupled to the closed ring of the aryl base, such as with toluene. Again solely by way of example and without limitation, a long-chain hydrocarbon may have a hydroxyl group bonded thereto.

“Hydroxyl” refers to the group —OH.

“Phosphate” refers to the group —O—P(O)(OR′)₂, where each —OR′ independently is —OH; —O-aliphatic, such as —O-alkyl or —O-cycloalkyl; —O-aromatic, including both —O-aryl and —O-heteroaryl; —O-aralkyl; or —OR′ is —O⁻M⁺, where M⁺ is a counter ion with a single positive charge. Each M⁺ may be an alkali ion, such as K⁺, Na⁺, Li⁺; an ammonium ion, such as ⁺N(R″)₄ where each R″ independently is H, aliphatic, heterocyclyl or aryl; or an alkaline earth ion, such as [Ca²⁺]_{0.5}, [Mg²⁺]_{0.5}, or [Ba²⁺]_{0.5}. Phosphonooxyalkyl refers to the group -alkyl-phosphate, such as, for example, —CH₂OP(O)(OH)₂, or a salt thereof, such as —CH₂OP(O)(O⁻Na⁺)₂, and (((dialkoxyphosphoryl)oxy)alkyl) refers to the dialkyl ester of a phosphonooxyalkyl group, such as, for example, —CH₂OP(O)(O-tert-butyl)₂.

### II. Compositions

The present invention concerns microbial compositions, such as suspensions of bacterial cells and/or spores, that are concentrated for storage and/or shipping in combination with a preservative system. Such compositions typically are then diluted by an end user to provide a suitable, ready-to-use (RTU) composition that is diluted sufficiently for a desired end use. For example, microbial compositions may be concentrated by at least 5 times to at least 250 times the concentration typically required for an end-use application, stored and/or shipped, and then diluted back to a useful composition concentration, such as a 5-fold to at least a 250-fold dilution as suitable for the intended end-use application.

A preservative system for stabilizing microbial suspensions, particularly spore-forming microbes, such as bacterial cells or spores, also is disclosed. Certain disclosed embodiments concern a concentrated bacterial suspension that, when diluted with water, retains stability and preservation over time under ambient conditions. When concentrated, stabilization is provided by a humectant solvent system that effectively solvates plural preservatives, with certain embodiments using a preservative composition comprising at least 3 different preservatives. Disclosed preservatives include, by way of example and without limitation, a paraben preservative, an isothiazolone preservative, and a quaternary ammonium preservative. When disclosed compositions are diluted with water, the preservatives also are diluted to allow partial to full solubilization.

Certain disclosed concentrated bacterial suspensions comprise bacterial cells or spores, a humectant solvent, a preservative system, optionally a buffering system, such as phosphate salts or ethylenediaminetetraacetic acid (EDTA), optionally a surfactant, typically a non-ionic surfactant, which slows or eliminates paraben-induced spore aggregation, optionally Tween 80, with water added in an amount as low as possible but sufficient to solubilize any necessarily solubilized components, such as buffering salts. Particular components and relative amounts therefore used to form embodiments of concentrated, preserved and stabilized spore-forming microbial compositions, are described in more detail below.

A. Bacterial Cells/Spores

Disclosed embodiments of the present invention can be used with substantially any spore-forming organisms, such as fungi and bacteria, including aerobic bacteria, such as Bacillus and Paenibacillus, and anaerobic species, such as Clostridium. A person of ordinary skill in the art will appreciate that any species or strain, or combinations of species or strains, of spore-forming microbials can be used to form concentrated, preserved and stabilized spore-forming microbial compositions according to the present invention.

Aspects of the present invention are illustrated primarily with reference to Bacillus bacterial cells or spores, including powdered spores as a culture source, and more particularly combinations of different species of Bacillus bacterial cells or spores. Bacilli are a genus of gram-positive bacteria. There are at least 266 names species of Bacillus, and the present invention is useful for forming stabilized systems comprising any such Bacillus species. These include B. acidiceler, B. acidicola, B. acidiproducens, B. acidocaldarius, B. acidoterrestris, B. aeolius, B. aerius, B. aerophilus, B. agaradhaerens, B. agri, B. aidingensis, B. akibai, B. alcalophilus, B. algicola, B. alginolyticus, B. alkalidiazotrophicus, B. alkalinitrilicus, B. alkalisediminis, B. alkalitelluris, B. altitudinis, B. alveayuensis, B. alvei, B. amyloliquefaciens, B. a. subsp. Amyloliquefaciens, B. a. subsp. Plantarum, B. aminovorans, B. amylolyticus, B. andreesenii, B. aneurinilyticus, B. anthracis, B. aquimaris, B. arenosi, B. arseniciselenatis, B. arsenicus, B. aurantiacus, B. arvi, B. aryabhattai, B. asahii, B. atrophaeus, B. axarquiensis, B. azotofixans, B. azotoformans, B. badius, B. barbaricus, B. bataviensis, B. beijingensis, B. benzoevorans, B. beringensis, B. berkeleyi, B. beveridgei, B. bogoriensis, B. boroniphilus, B. borstelensis, B. brevis Migula, B. butanolivorans, B. canaveralius, B. carboniphilus, B. cecembensis, B. cellulosilyticus, B. centrosporus, B. cereus, B. chagannorensis, B. chitinolyticus, B. chondroitinus, B. choshinensis, B. chungangensis, B. cibi, B. circulans, B. clarkii, B. clausii, B. coagulans, B. coahuilensis, B. cohnii, B. composti, B. curdlanolyticus, B. cycloheptanicus, B. cytotoxicus, B. daliensis, B. decisifrondis, B. decolorationis, B. deserti, B. dipsosauri, B. drentensis, B. edaphicus, B. ehimensis, B. eiseniae, B. enclensis, B. endophyticus, B. endoradicis, B. farraginis, B. fastidiosus, B. fengqiuensis, B. firmus, B. Plexus, B. foraminis, B. fordii, B. formosus, B. fortis, B. fumarioli, B. funiculus, B. fusiformis, B. galactophilus, B. galactosidilyticus, B. galliciensis, B. gelatini, B. gibsonii, B. ginsengi, B. ginsengihumi, B. ginsengisoli, B. glucanolyticus, B. gordonae, B. gottheilii, B. graminis, B. halmapalus, B. haloalkaliphilus, B. halochares, B. halodenitrificans, B. halodurans, B. halophilus, B. halosaccharovorans, B. hemicellulosilyticus, B. hemicentroti, B. herbersteinensis, B. horikoshii, B. horneckiae, B. horti, B. huizhouensis, B. humi, B. hwajinpoensis, B. idriensis, B. indicus, B. infantis, B. infernus, B. insolitus, B. invictae, B. iranensis, B. isabeliae, B. isronensis, B. jeotgali, B. kaustophilus, B. kobensis, B. kochii, B. kokeshiiformis, B. koreensis, B. korlensis, B. kribbensis, B. krulwichiae, B. laevolacticus, B. larvae, B. laterosporus, B. lautus, B. lehensis, B. lentimorbus, B. lentus, B. licheniformis, B. ligniniphilus, B. litoralis, B. locisalis, B. luciferensis, B. luteolus, B. luteus, B. macauensis, B. macerans, B. macquariensis, B. macyae, B. malacitensis, B. mannanilyticus, B. marisflavi, B. marismortui, B. marmarensis, B. massiliensis, B. megaterium, B. mesonae, B. methanolicus, B. methylotrophicus, B. migulanus, B. mojavensis, B. mucilaginosus, B. muralis, B. murimartini, B. mycoides, B. naganoensis, B. nanhaiensis, B. nanhaiisediminis, B. nealsonii, B. neidei, B. neizhouensis, B. niabensis, B. niacin, B. novalis, B. oceanisediminis, B. odysseyi, B. okhensis, B. okuhidensis, B. oleronius, B. oryzaecorticis, B. oshimensis, B. pabuli, B. pakistanensis, B. pallidus, B. pallidus, B. panacisoli, B. panaciterrae, B. pantothenticus, B. parabrevis, B. paraflexus, B. pasteurii, reclassified as Sporosarcina pasteurrii, other Sporosarcina species, B. patagoniensis, B. peoriae, B. persepolensis, B. persicus, B. pervagus, B. plakortidis, B. pocheonensis, B. polygoni, B. polymyxa, B. popilliae, B. pseudalcalophilus, B. pseudofirmus, B. pseudomycoides, B. psychrodurans, B. psychrophilus, B. psychrosaccharolyticus, B. psychrotolerans, B. pulvifaciens, B. pumilus, B. purgationiresistens, B. pycnus, B. qingdaonensis, B. qingshengii, B. reuszeri, B. rhizosphaerae, B. rigui, B. runs, B. safensis, B. salarius, B. salexigens, B. sahphilus, B. schlegelii, B. sediminis, B. selenatarsenatis, B. selenitireducens, B. seohaeanensis, B. shacheensis, B. shackletonii, B. siamensis, B. silvestris, B. simplex, B. siralis, B. smithii, B. soli, B. solimangrovi, B. solisalsi, B. songklensis, B. sonorensis, B. sphaericus, B. sporothermodurans, B. stearothermophilus, B. stratosphericus, B. subterraneus, B. subtilis, B. s. subsp. Inaquosorum, B. s. subsp. Spizizenii, B. s. subsp. Subtilis, B. taeanensis, B. tequilensis, B. thermantarcticus, B. thermoaerophilus, B. thermoamylovorans, B. thermocatenulatus, B. thermocloacae, B. thermocopriae, B. thermodenitrificans, B. thermoglucosidasius, B. thermolactis, B. thermoleovorans, B. thermophilus, B. thermoruber, B. thermosphaericus, B. thiaminolyticus, B. thioparans, B. thuringiensis, B. tianshenii, B. trypoxylicola, B. tusciae, B. validus, B. vallismortis, B. vedderi, B. velezensis, B. vietnamensis, B. vireti, B. vulcani, B. wakoensis, B. xiamenensis, B. xiaoxiensis, B. zanthoxyli, B. zhanjiangensis.

Particular Bacillus species suitable for practicing the present invention include Bacillus coagulans, Bacillus pumilus, Bacillus thuringiensis, Bacillus inaquosorum, Bacillus mojavensis, Bacillus megaterium, Bacillus globigii, Bacillus subtilis, Bacillus simplex, Bacillus licheniformis and/or Bacillus amyloliquefaciens.

The amount or concentration of bacterial cells in a sample may be stated with reference to the number of colony forming units (CFUs) present in a composition, such as a bacterial suspension. A CFU is a unit used in microbiology to estimate the number of viable bacteria or fungal cells in a sample, as will be understood by a person of ordinary skill in the art. Counting colony-forming units requires culturing the microbes and then counting viable cells.

For the present invention and with reference to the concentrate, the CFUs can vary from 1×10³ to 4×10¹¹, more typically from 1×10⁸ to 5×10¹⁰ CFUs. With reference to the RTU, the CFUs can vary from at least as low as 1×10² CFUs to at least as high as 4×10¹⁰ CFUs, and more typically between about 1×10⁷ to 1 or 1×10⁹ CFUs.

B. Solvent

Concentrated microbial compositions according to the present invention, such as bacterial suspensions of cells and/or spores, typically are formulated with a solvent, particularly a humectant solvent, i.e. a solvent selected to reduce moisture loss from the composition. Suitable humectant solvents according to the present invention typically are polar organic compounds. More particularly, suitable humectant solvents typically are alcohols, diols, triols, glycols (a diol, and more particularly a compound having two hydroxyl groups on adjacent carbon atoms) and glycerol (propane-1,2,3-triol), a particular example of a suitable triol. Particularly suitable humectant solvents for use with the present invention are the alkylene glycols, where “alkylene” is typically C₁₋₁₀ alkyl, more typically C₁₋₅ alkyl, such as ethylene glycol and propylene glycol (propane-1,2-diol). Propylene glycol was used in certain representative examples as discussed in more detail below in the Examples.

The humectant solvent is used to form a preservative composition, also referred to herein as Prep B. The humectant solvent also may be used to form microbial suspension compositions. In practice, a Prep B composition is usually formed and stored, a bacterial suspension composition is formed immediately prior to use, and then appropriate amounts of the Prep B composition and bacterial suspension composition are combined to form a concentrated, stabilized and preserved bacterial suspension composition. Accordingly, the amount of humectant solvent used to form disclosed compositions can be considered solely with reference to the Prep B composition concentrations, and/or with reference to the concentration in combined stabilized, preserved microbial suspension compositions. In general, the concentration of solvent or solvents used, such as the humectant solvent and/or water, are as low as possible that still allows solubilization of composition components that are intended to be solubilized, such as buffering salts. In general, unless stated otherwise, amounts herein are specified as weight percents (wt. %). The humectant solvent may be used in an upper range up to about 99%. For example, a 10× formulation having a minimum threshold preservation levels, no buffer/salt, no threshold spore-forming microbe such as Bacillus, and no water, could include 99% humectant solvent. However, humectant solvents used to formulate Prep B compositions are more generally used in an amount ranging from 35% to 65%, and more typically 42%-43%. With reference to combined stabilized, preserved microbial suspension compositions, that is a Prep B composition combined with a microbial suspension, the humectant solvent is used in an amount of about 35% to about 45%. Certain disclosed exemplary embodiments comprised about 41% total weight percent propylene glycol for a 250× concentrate, and about 36% propylene glycol for a 100× concentrate.

C. Preservatives

The present invention typically uses a combination of preservatives to form Prep B compositions and concentrated stabilized and preserved microbial suspensions, such as a combination of at least 2 different preservatives up to at least 5 different preservatives. Exemplary disclosed compositions used a combination of 3 different classes of preservatives, that is a first preservative, a second preservative and a third preservative, where the first, second and third preservatives are different.

Suitable preservatives include, by way of example and without limitation:

isothiazolinone preservatives, such as a 1,2-benzoisothiazolin-3-one (BIT), methylisothiazolinone (MIT), methylchloroisothiazolinone (CMIT), and combinations thereof. The structures of these preservatives are provided below:

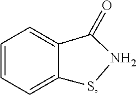

1,2-Benzoisothiazolin-3-one (1,2-Benzothiazol-3(2H)-one)

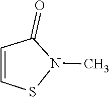

Methylisothiazolinone (2-Methyl-1,2-thiazol-3(2H)-one)

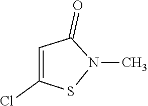

Methylchloroisothiazolinone (5-Chloro-2-methyl-1,2-thiazol-3(2H)-one)

Certain representative embodiments discussed in the Examples used 1,2-benzoisothiazolin-3-one. A suitable 1,2-benzoisothiazolin-3-one preservative is commercially available as Proxel GXL from Lonza as a 15%-20% aqueous dipropylene glycol solution comprising 1,2-benzoisothiazolin-3-one and 5% to 10% sodium hydroxide. Isothiazolinone preservatives are used in any effective amount, as will be understood by a person of ordinary skill in the art, but generally are used in an amount of greater than 0% to less than about 1%, more typically 0.05% to less than 0.5%, of the active preservative in RTU formulations. Disclosed representative 250× concentrate embodiments used about 10% of a Proxel GXL comprising 20% active preservative, which equates to about 2% active preservative in the 250× concentrate. Representative 100× concentrate compositions used 4% Proxel GXL, equating to 0.8% active preservative;

quaternary ammonium preservatives, typically having a formula R¹R²R³R⁴N⁺X⁻, where R¹-R⁴ are the same or different, and are, by way of example and without limitation, independently selected from H, alkyl, such as C₁-C₂₀ alkyl, and aryl, such as phenyl or benzyl. Particular examples of quaternary ammonium preservatives include benzalkonium chloride, cetyl pyridinium chloride (commercially available as Ceepryn), and octylphenoxyethoxyethyl dimethyl benzyl ammonium chloride (commercially available as Phemerol). Representative embodiments discussed in the Examples used benzalkonium chloride as the preservative. A suitable benzalkonium chloride preservative is commercially available from Stepan as Stepenquat 65NF, a composition comprising 50% benzalkonium chloride preservative and 2-5% ethanol. Quaternary ammonium preservatives are generally used in the RTU formulation in an amount of about greater than 0% to 1% of the active preservative, more typically 0.025% to 0.5%. Disclosed representative 250× concentrate embodiments used about 5% of such commercially available composition, which equates to about 2.5% active benzalkonium chloride preservative. Representative 100× concentrate compositions used 2% of such commercially available benzalkonium chloride preservative composition, equating to 1.25% active preservative;

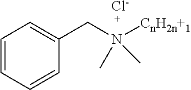

Benzalkonium chloride (N-Alkyl-N-benzyl-N,N-dimethylammonium chloride)

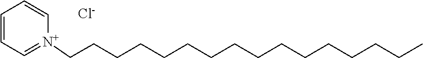 [SMILES: CCCCCCCCCCCCCCCC[n+]1ccccc1 ; [Cl-]]

Cetyl pyridinium chloride (1-hexadecylpyridin-1-ium chloride)

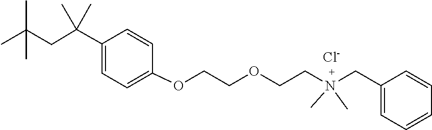

octylphenoxyethoxyethyl dimethyl benzyl ammonium chloride and

paraben-class preservatives, where paraben refers to a para-hydroxybenzoate, shown below,

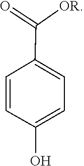

With reference to this formula, R is an alkyl group, typically C₁-C₂₀ alkyl, more typically C₁-C₅ alkyl, such as methyl, ethyl, or propyl, with methylparaben (R═CH₃) and propylparaben (R═C₃H₇) being particular examples of suitable preservatives. Propylparaben was used in certain representative examples, as discussed below in the Examples. Paraben preservatives are generally used in the RTU formulation in an amount of about greater than 0% to 1% of the active preservative, more typically 0.025% to 0.5%. Disclosed representative 250× concentrate embodiments used 5% propylparaben, whereas representative 100× concentrate compositions used 2% propylparaben;

Certain disclosed representative 250× Prep B compositions comprised, consisted of or consisted essentially of a particular combination of preservatives and amounts, namely 20% to 40%, more typically 25% to 35% Proxel GXL, 10%-25% Stepenquat 65 NF, more typically 12%-18%, and 10%-25% propylparaben, more typically 12%-18%, in a composition comprising 30% to 70% humectant solvent, more typically 35% to 55%, such as propylene glycol.

D. Surfactants, Processing Aids and Defoamers

A person of ordinary skill in the art will appreciate one, or combination, of surfactants can be used to make suitable compositions according to the present invention, preferably no-foaming or low-foaming surfactants. Suitable classes of surfactants are nonionic surfactants, anionic surfactants, cationic surfactants, amphoteric surfactants, zwitterionic surfactants, and mixtures thereof. Suitable surfactants can be emulsifiers and/or detersive surfactants. Mixtures of emulsifiers and detersive surfactants also can be used. When a surfactant containing one, or more, aliphatic alkyl group is used, it is preferred that it contain relatively short alkyl chains of from about 5 to about 14 carbon atoms. Suitable nonionic surfactants are polyethylene glycol-polypropylene glycol block copolymers, such as Puronic® and Pluronic R® surfactants from BASF; Tetronic® and Tetronic R® surfactants from BASF, ethoxylated branched aliphatic diols such as Surfynol® surfactants from Air Products; ethoxylated alkyl phenols, such as Igepal® surfactants from Rhone-Poulenc; ethoxylated aliphatic alcohols and carboxylic acids; polyethylene glycol diesters of fatty acids; fatty acid esters of ethoxylated sorbitans; and mixtures thereof. Suitable anionic surfactants are dialkyl sulfosuccinate, alkylarylsulfonate, fatty alcohol sulfate, paraffin sulfonate, alkyl sarcosinate, alkyl isethionate salts having suitable cations, e.g., sodium, potassium, alkanol ammonium, etc., and mixtures thereof. Suitable amphoteric surfactants are the betaines. Suitable surfactants may have hydrophilic groups situated between hydrophobic chains, such as Pluronic R® surfactants, Surfynol surfactants, polyethylene glycol diesters of fatty acids, fatty acid esters of ethoxylated sorbitans, dialkyl sulfosuccinate, di(C8-C12 alkyl)di(C1-C2 alkyl)ammonium halides, and mixtures thereof; or surfactants that have hydrophobic chains situated between hydrophilic groups, such as Pluronic surfactants; and mixtures thereof. Particular suitable surfactants include: a sorbitan ester (SPANS), including but not limited to sorbitan monolaurate, sorbitan monostearate, sorbitan tristearate, or any sorbitan+fatty acid (derived or otherwise); polysorbates (TWEENS), including but not limited to, polysorbate 20, 40, 60, 80, etc.; tritons (polyoxyethylene glycol octylphenol ethers); ethoxylates; nonoxynol; ethoquad; block copolymers of PEG and PPG, such as poloxamers; quaternary ammonium surfactants; monoalkyl ammonium surfactants with hydrophobic groups (R groups), such as cetyl, stearyl, lauryl, biphenyl, etc., including benzalkonium chlorides and centrimonium bromide; dialkyl ammonium surfactants again with hydrophobic groups; esterquats, as above with fatty esters in place of the R groups; ethoxylated ammonium; and betaine surfactants. Suitable surfactants also may include sulfonates, such as AEOS, SLS, SLES, and lignosulfonates and other anionic surfactants, both alone and in combination. Suitable exemplary biosurfactants include lipopeptide surfactant (cyclic or non-cyclic), glycolipids, polymeric biosurfactants, lipopolysaccharidic surfactants, and mannosylerythritol biosurfactants. Particular examples of suitable biosurfactants include surfactins, fengycins, iturins, lichenycins, mycosubtilins, bacilomycin, any other Bacillus-derived biosurfactant, rhamnolipids, bile salts, lecithin, sophorolipids, emulsans, trehalolipids, liposan, and alasan.

The Environmental Protection Agency (EPA) provides additional examples of suitable surfactants on its Safer Choice list, at (https://www.epa.gov/saferchoice/saferingredients#searchList), and such surfactants include: (Z)-7-Hexadecenoic acid (2416-19-5), (Z)-9-Hexadecenoic acid (373-49-9), β-Alanine, N-(2-carboxyethyl)-N-[3-(decyloxy)propyl]-, sodium salt (1:1) (64972-19-6),1-Dodecanesulfonic acid, hydroxy-, sodium salt (128824-30-6), 1-Hexadecanaminium, N-(carboxymethyl)-N,N-dimethyl-, inner salt(693-33-4),1-propanaminium, 3-amino-N-(carboxymethyl)-N,N-dimethyl-, N—(C8-18 and C18-unsatd. acyl) derivs., inner salts(147170-44-3), 1-Propanaminium, 3-amino-N-(carboxymethyl)-N,N-dimethyl-, N-coco acyl derivatives, chlorides, sodium salts (61789-39-7), 1-Propanaminium, 3-amino-N-(carboxymethyl)-N,N-dimethyl-, N-coco acyl derivs., inner salts(61789-40-0), 1-Propanaminium, N-(carboxymethyl)-N,N-dimethyl-3-[(1-oxodecyl)amino]-, inner salt(73772-45-9), 1-Propanaminium, N-(carboxymethyl)-N,N-dimethyl-3-[(1-oxooctyl)amino]-, inner salt(73772-46-0), 1H-Imidazole-1-propanoic acid, 2-heptyl-2,3-dihydro-3-(2-hydroxyethyl)-, monosodium salt(68630-95-5), 2-Ethylhexyl-alpha-D-glucoside (125590-73-0), 2-Ethylhexyl-poly-D-glucosides (161074-93-7), 2-O-Rhamnopyranosyl-rhamnopyranosyl-3-hydroxyldecanoyl-3-hydroxydecanoate(4348-76-9), 9-Eicosenoic acid (506-31-0), half acetic acid, 2-chloro-, reaction products with 2-heptyl-4,5-dihydro-1H-imidazole-1-ethanol and sodium hydroxide (68608-64-0), acetic acid, chloro-, sodium salt, reaction products with 4,5-dihydro-2-undecyl-1H-imidazole-1-ethanol and sodium hydroxide (68608-66-2), Alcohols, C10-12, ethoxylated propoxylated (68154-97-2), defoamers; alcohols, C10-14, ethoxylated (66455-15-0), alcohols, C10-16, ethoxylated (68002-97-1), alcohols, C10-16, ethoxylated propoxylated (69227-22-1), alcohols, C11-14-iso-, C13-rich, ethoxylated (78330-21-9), alcohols, C11-15-secondary, ethoxylated (68131-40-8), alcohols, C12-13, ethoxylated (66455-14-9), alcohols, C12-14, ethoxylated (68439-50-9), alcohols, C12-14, ethoxylated propoxylated (68439-51-0), alcohols, C12-14-secondary, ethoxylated (84133-50-6), alcohols, C12-15 (63393-82-8), alcohols, C12-15, ethoxylated (68131-39-5), alcohols, C12-15, ethoxylated propoxylated (68551-13-3), alcohols, C12-15-branched and linear, ethoxylated propoxylated (120313-48-6), alcohols, C12-16, ethoxylated (68551-12-2), alcohols, C12-16, ethoxylated propoxylated (68213-24-1), alcohols, C12-18 (67762-25-8), alcohols, C12-18, ethers with polyethylene glycol mono-Bu ether (146340-16-1), alcohols, C12-18, ethoxylated (68213-23-0), alcohols, C14-15, ethoxylated (68951-67-7), alcohols, C16-18, ethoxylated (68439-49-6), alcohols, C6-10, ethoxylated (70879-83-3), alcohols, C6-10, ethoxylated propoxylated (68987-81-5), alcohols, C6-12, ethoxylated (68439-45-2), alcohols, C6-12, ethoxylated and propoxylated (68937-66-6), alcohols, C8-10, ethoxylated (71060-57-6), alcohols, C8-10, ethoxylated propoxylated (68603-25-8), half alcohols, C8-16, ethoxylated (71243-46-4), alcohols, C8-18, ethoxylated propoxylated (69013-18-9), half alcohols, C9-11, ethers with polyethylene glycol mono-Me ether (366009-01-0), alcohols, C9-11, ethoxylated (68439-46-3), alkyl(C12-C16)alcohol sulfate sodium salt (73296-89-6), amides, coco, N-[3-(dimethylamino)propyl], alkylation products with chloroacetic acid, sodium salts (70851-07-9), amines, C10-16-alkyldimethyl, N-oxides (70592-80-2), amines, C12-18-alkyldimethyl, N-oxides (68955-55-5), Amines, coco alkyl dihydroxyethyl, oxides(61791-47-7), ammonium cumene sulfonate (37475-88-0), ammonium lauryl sulfate (2235-54-3), ammonium xylenesulfonate (26447-10-9), benzene, (1-methylethyl)-, monosulfo deriv., sodium salt (1:1) (32073-22-6), benzenesulfonic acid, 4-C10-13-sec-alkyl derivs. (85536-14-7), benzenesulfonic acid, 4-C10-13-sec-alkyl derivs., sodium salts (127184-52-5), benzenesulfonic acid, C10-14-alkyl derivs., sodium salts (69669-44-9), benzenesulfonic acid, C10-16-alkyl derivs. (68584-22-5), benzenesulfonic acid, C10-16-alkyl derivs., magnesium salts (68584-26-9), benzenesulfonic acid, mono-C10-16-alkyl derivs., sodium salts (68081-81-2), half β-alanine, N-(2-carboxyethyl)-, N-coco alkyl derivs., disodium salts (90170-43-7), β-alanine, N-(2-carboxyethyl)-N-(2-ethylhexyl)-, sodium salt (1:1) (94441-92-6), half β-Alanine, N-(2-carboxyethyl)-N-[3-(octyloxy)propyl]-, sodium salt (1:1)(64972-18-5), betaines, C10-16-alkyl(2-hydroxy-3-sulfopropyl)dimethyl (72869-77-3), betaines, coco alkyldimethyl (68424-94-2), butanedioic acid, sulfo-, mono(C10-C16)alkyl ethoxylated ester, disodium salt (68815-56-5), butyl D-glucoside (31387-97-0), butyl poly-D-glucosides (510758-10-8), C10-13 Alkyl benzenesulfonic acid, sodium salts (90194-45-9), C10-16 Alkyl-poly-D-glucosides (110615-47-9), C12-14 Alkyl-poly-D-glucosides (157707-88-5), C12-16 Alkyl-poly-D-glucosides (510759-65-6), C6-12 Alkyl-poly-D-glucosides (113976-90-2), C8-10 Alkyl-poly-D-glucosides (161074-97-1), C8-16 Alkyl-poly-D-glucosides (141464-42-8), C9-11 Alkyl-poly-D-glucosides (132778-08-6), calcium xylene sulfonate (28088-63-3), capric dimethyl amine oxide (2605-79-0), castor oil, ethoxylated (61791-12-6), castor oil, hydrogenated, ethoxylated (61788-85-0), castor oil, sulfated, sodium salt (68187-76-8), cetamine oxide (7128-91-8), cetearyl glucoside (246159-33-1), cocamidopropyl hydroxysultaine (68139-30-0), cocamidopropyl hydroxysultaine (70851-08-0), cocamidopropylamine oxide (68155-09-9), cocamine oxide (61788-90-7), coconut fatty acids, ethoxylated (61791-29-5), coconut oil, sodium salt (68814-96-0), cyclocarboxypropyloleic acid (53980-88-4), D-Glucitol, 1-deoxy-1-(methylamino)-, N—(C16-18 and C18-unsatd. acyl) derivs. (1591782-99-8), D-Glucitol, 1-deoxy-1-(methylamino)-, N—C8-10 acyl derivs. (1591782-62-5), D-Glucitol, 1-deoxy-1-(methylamino)-, N-coco acyl derivs. (1591783-13-9), D-Glucopyranose, oligomeric, 6-(dihydrogen 2-hydroxy-1,2,3-propanetricarboxylate), 1-(coco alkyl) ethers, sodium salts(151911-51-2), D-Glucopyranose, oligomeric, C10-16-alkyl glycosides, 2-hydroxy-3-sulfopropyl ethers, sodium salts (742087-49-6), D-Glucopyranose, oligomeric, heptyl glycosides (1627851-18-6), Decanoic acid (334-48-5).

Suitable processing aids and additives include: cecanoic acid, potassium salt (1:1) (13040-18-1), decyl glucoside (54549-25-6), decyl glucoside (58846-77-8), decyl, octyl-poly-D-glucosides (68515-73-1), decyl-poly-D-glucosides (161074-85-7), decylbenzenesulfonic acid (1322-98-1), diethylene glycol momolauryl ether sodium sulfate (3088-31-1), half disodium 2-sulfolaurate (38841-48-4), half disodium 2-sulfomyristate (5802-92-6), disodium cocoyl glutamate (68187-30-4), half disodium lauriminodipropionate (3655-00-3), distearoylethyl hydroxyethylmonium methosulfate (32208-04-1), ocosanoic acid (112-85-6), dodecanoic acid (143-07-7), dodecanoic acid, sodium salt (629-25-4), dodecene-1-sulfonic acid, sodium salt (30965-85-6), dodecyl alcohol, ethoxylated (9002-92-0), dodecyl triethylene glycol ether (3055-94-5), dodecyl-beta-D-glucoside(59122-55-3), dodecylbenzene sulfonic acid (27176-87-0), Eicosanoic acid (506-30-9), ethanaminium, 2-hydroxy-N,N-bis(2-hydroxyethyl)-N-methyl-, esters with C16-18 and C18-unsatd. fatty acids, methyl sulfates (salts) (157905-74-3), ethanaminium, 2-hydroxy-N-(2-hydroxyethyl)-N,N-dimethyl-, diesters with C16-18 and C18-unsatd. fatty acids, Me sulfates (193635-82-4), ethanaminium, 2-hydroxy-N-(2-hydroxyethyl)-N,N-dimethyl-, esters with C16-18 and C18-unsatd. fatty acids, chlorides (1079184-43-2), ethanaminium, N,N-dimethyl-2-[(1-oxohexadecyl)oxy]-N-[2-[(1-oxohexadecyl)oxy]ethyl]-, chloride (1:1) (97158-31-1), ethanaminium, N,N-dimethyl-2-[(1-oxooctadecyl)oxy]-N-[2-[(1-oxooctadecyl)oxy]ethyl]-, chloride (67846-68-8), ethylene glycol monopalmitate (4219-49-2), Ethylene glycol monostearate (111-60-4), fatty acids, C10-16 (68002-90-4), fatty acids, C10-20 and C16-18-unsatd., reaction products with triethanolamine, di-Me sulfate-quaternized (91995-81-2), fatty acids, C12-18 (67701-01-3), fatty acids, C12-18, methyl esters, sulfonated, sodium salts (149458-07-1), fatty acids, C12-20, reaction products with triethanolamine, di-mesulfate-quaternized (91032-11-0), fatty acids, C14-18 and C16-18-unsatd. (67701-06-8), fatty acids, C14-18 and C16-18-unsatd., sodium salts (67701-11-5), fatty acids, C14-22 and C16-22-unsatd. (68002-85-7), fatty acids, C16-C18 and C18 unsaturated, branched and linear (68955-98-6), Fatty acids, C16-18 (67701-03-5), fatty acids, C16-18 and C18-unsatd. (67701-08-0), Fatty acids, C16-18 and C18-unsatd., esters with polyethylene glycol mono-Me ether518299-31-5), half fatty acids, C16-18, ethoxylated (68989-61-7), fatty acids, C18 and C18-unsatd. (68002-84-6), fatty acids, C8-18 and C18-unsaturated (67701-05-7), fatty acids, C8-18 and C18-unsaturated, sodium salts (67701-10-4), fatty acids, coco (61788-47-4), fatty acids, coco, heavy fractions (68937-85-9), fatty acids, coco, sodium salts (61789-31-9), fatty acids, coconut oil, potassium salts (61789-30-8), fatty acids, coconut oil, sulfoethyl esters, sodium salts (61789-32-0), fatty acids, olive-oil, potassium salts (68154-77-8), fatty acids, olive-oil, sodium salts (61789-88-6), fatty acids, palm kernel-oil, methyl esters, sulfonated, sodium salts (68440-13-1), fatty acids, palm kernel-oil, sodium salts (61789-89-7), fatty acids, palm-oil (68440-15-3), fatty acids, palm-oil, sodium salts (61790-79-2), fatty acids, safflower-oil, sodium salts (68440-19-7), fatty acids, tall oil, sodium salt (61790-45-2), fatty acids, tall-oil (61790-12-3), fatty acids, tall-oil, potassium salts (61790-44-1), fatty acids, tallow (61790-37-2), fatty acids, tallow, hydrogenated, compds. with triethanolamine (68605-97-0), fatty acids, tallow, sodium salts (8052-48-0), gardol (137-16-6), glycerides, C14-18 mono- and di-(67701-33-1), glycerides, C16-18 and C18-unsatd. mono- and di-(68424-61-3), glycerides, C16-18 mono- and di-(85251-77-0), glycerides, C8-10 mono-, di- and tri-(91744-32-0), glycerides, C8-18 and C18-unsaturated mono- and di-acetates (91052-13-0), glycerides, castor-oil mono-, hydrogenated, acetates (736150-63-3), glycerine oleate (37220-82-9), half glyceryl monolaurate (142-18-7), glyceryl monooleate (111-03-5), glyceryl monooleate [NF](25496-72-4), glyceryl monostearate (123-94-4), glyceryl monostearate (31566-31-1), glyceryl stearates(11099-07-3), glycine, N-[2-(carboxymethoxy)ethyl]-N-[2-[(1-oxododecyl)amino]ethyl]-, disodium salt (68298-20-4), glycine, N-methyl-, N-coco acyl derivs. (68411-97-2), glycol distearate (627-83-8), heptadecanoic acid (506-12-7), hexanoic acid (142-62-1), hexyl D-glucoside(54549-24-5), hexyl poly(oxyethylene) ether (31726-34-8), imidazolium compounds, 1-[2-(2-carboxyethoxy)ethyl]-1(or 3)-(2-carboxyethyl)-4,5-dihydro-2-norcoco alkyl, hydroxides, disodium salts (68604-71-7), imidazolium compounds, 1-[2-(carboxymethoxy)ethyl]-1-(carboxymethyl)-4,5-dihydro-2-norcoco alkyl, inner salts, disodium salts (68650-39-5), half L-alanine, N-coco acyl derivs., sodium salts (90170-45-9), lauramidopropyl betaine (4292-10-8), Lauramidopropylamine oxide (61792-31-2), laureth-11 carboxylic acid, sodium salt (33939-64-9), lauryl glucoside (27836-64-2), lauryl hydroxysultaine (13197-76-7), lauryldimethylbetaine (683-10-3), lecithins (8002-43-5), lignoceric acid (557-59-5), linoleic acid (60-33-3), linolenic acid (463-40-1), magnesium lauryl sulfate (3097-08-3), half magnesium stearate (557-04-0), myristamido propylamine oxide (67806-10-4), myristamidopropyl betaine (59272-84-3), myristoleic acid (544-64-9), myristyl alcohol, ethoxylated (27306-79-2), myristyl betaine (2601-33-4), N,N-Dimethyl-1-tetradecanamine-N-oxide (3332-27-2), N,N-dimethyldodecylamine oxide(1643-20-5), half N-(3-Alkyl(C12-C15)oxypropyl)-3-iminodipropionic acid, monosodium salt(68608-69-5), n-Octylpolyoxyethylene (27252-75-1), N-Octylpyrrolidone (2687-94-7), octadecanoic acid, 12-hydroxy-(106-14-9), octadecanoic acid, 2-ethylhexyl ester (22047-49-0), octanoic acid (124-07-2), half octanoic acid, monoester with 1,2,3-propanetriol (26402-26-6), octanoic acid, potassium salt (764-71-6), octanoic acid, reaction products with 2-[(2-aminoethyl)amino]ethanol, acrylic acid alkylated (1:2), disodium salts (68815-55-4), octyl-beta-D-glucoside (29836-26-8), octyldimethylamine oxide (2605-78-9), octyldimethylbetaine (27593-14-2), oleic acid (112-80-1), oleic acid, sodium salt (143-19-1), half oxirane, 2-methyl-, polymer with oxirane, mono(2-propylheptyl) ether (166736-08-9), oxirane, methyl-, polymer with oxirane, mono(2-ethylhexyl) ether (64366-70-7), palmitic acid (57-10-3), palmitoleic acid (2091-29-4), and poloxalene (9003-11-6).

Suitable exemplary defoamers include: poly(oxy-1,2-ethanediyl), α-(3-carboxy-1-oxo-3-sulfopropyl)-.omega.-(dodecyloxy)-, disodium salt (39354-45-5), poly(oxy-1,2-ethanediyl), α-(carboxymethyl)-.omega.-(octyloxy)-(53563-70-5), poly(oxy-1,2-ethanediyl), α-decyl-.omega.-hydroxy-(26183-52-8), poly(oxy-1,2-ethanediyl), .alpha.-sulfo-.omega.-hydroxy-, C10-16-alkyl ethers, ammonium salts (67762-19-0),poly(oxy-1,2-ethanediyl), α-sulfo-.omega.-hydroxy-, C6-10-alkyl ethers, ammonium salts (68037-05-8), half poly(oxy-1,2-ethanediyl), α-(2-ethylhexyl)-omega-hydroxy-(26468-86-0), poly(oxy-1,2-ethanediyl), alpha-(2-propylheptyl)-omega-hydroxy-(160875-66-1), poly(oxy-1,2-ethanediyl), alpha-(3-carboxy-1-oxosulfopropyl)-omega-hydroxy-, C10-12-alkyl ethers, disodium salts (68954-91-6), poly(oxy-1,2-ethanediyl), α-(carboxymethyl)-omega-hydroxy-, C12-13-alkyl ethers (70750-17-3), poly(oxy-1,2-ethanediyl), α-(carboxymethyl)-omega-hydroxy-,C12-14-alkyl ethers (220622-96-8), poly(oxy-1,2-ethanediyl), α-butyl-omega-(octyloxy)-(109075-72-1), poly(oxy-1,2-ethanediyl), alpha-hydro-omega-hydroxy-, mono-C8-10-alkyl ethers, ethers with 1,2-dodecanediol (1:1) (501019-91-6), poly(oxy-1,2-ethanediyl), alpha-tridecyl-omega-hydroxy-, branched (69011-36-5), poly(oxy-1,2-ethanediyl), alpha-undecyl-omega-hydroxy-, branched and linear(127036-24-2), polyethylene glycol distearate (9005-08-7), polyethylene glycol mono(tridecyl) ether (24938-91-8), polyethylene glycol mono-C10-16-alkyl ether sulfate sodium salt (68585-34-2), polyethylene glycol mono-C12-14-alkyl ether sulfate sodium salt (68891-38-3), polyethylene glycol monocetyl ether (9004-95-9), polyethylene glycol monoisotridecyl ether(9043-30-5), polyethylene glycol monoleyl ether (9004-98-2), polyethylene glycol stearate (9004-99-3), half polyoxyethylene dioleate (9005-07-6), polyoxyethylene monoleate (9004-96-0), polyoxyethylene monooctadecyl ether (9005-00-9), polyoxyethylene monoundecyl ether (34398-01-1), polyoxyethylene sorbitan trioleate (9005-70-3), polysorbate 80 (9005-65-6), potassium acrylinoleate (68127-33-3), potassium babassuate (226993-76-6), potassium cocoyl glycinate (301341-58-2), potassium dodecanoate (10124-65-9), potassium dodecylbenzene sulfonate (27177-77-1), half potassium heptadecanoate (17378-36-8), potassium linoleate (3414-89-9), potassium myristate (13429-27-1), potassium oleate (143-18-0), potassium palmitate (2624-31-9), potassium palmitoleate (593-29-3), potassium toluenesulfonate (16106-44-8), potassium xylene sulfonate (30346-73-7), propanoic acid, 2-hydroxy-, 2-(C10-16-alkyloxy)-1-methyl-2-oxoethyl ester(910661-93-7), propanoic acid, 3-[2-(2-heptyl-4,5-dihydro-1H-imidazol-1-yl)ethoxy]-, sodium salt (68877-55-4), quaternary ammonium compounds, bis(hydroxyethyl)methyltallow alkyl, ethoxylated, methyl sulfates (salts) (73138-81-5), quaternary ammonium compounds, coco-alkylbis(hydroxyethyl)methyl, ethoxylated, chlorides (61791-10-4), rhamnopyranosyl-3-hydroxydecanoyl-3-hydroxydecanoate (37134-61-5), sodium (Z)-hexadec-9-enoate (6610-24-8), sodium 1-methoxy-1-oxohexadecane-2-sulphonate (4016-24-4), sodium 1-octanesulfonate (5324-84-5), sodium cocoyl glutamate (68187-32-6), sodium cumene sulfonate (28348-53-0), sodium decanoate (1002-62-6), half sodium decylglucosides hydroxypropylsulfonate (742087-48-5), sodium dodecylpoly(oxyethylene) sulfate (9004-82-4), sodium ethasulfate (126-92-1), sodium hexadecyl sulfate (1120-01-0), half sodium laurimino dipropionate (14960-06-6), sodium lauroyl glutamate(29923-31-7), sodium lauroyl lactylate (13557-75-0), sodium lauroyl methyl isethionate (928663-45-0), sodium lauryl benzene sulfonate (25155-30-0), sodium lauryl sulfate (151-21-3), sodium lauryl sulfoacetate (1847-58-1), sodium lauryl trioxyethylene sulfate (13150-00-0), sodium linoleate (822-17-3), sodium methyl cocoyl taurate (61791-42-2), sodium myristate (822-12-8), sodium myristol sarcosinate (30364-51-3), half sodium myristyl sulfate (1191-50-0), sodium N-methyl-N-oleoyltaurate (137-20-2), sodium octanoate (1984-06-1), sodium octyl sulfate (142-31-4), sodium palmitate (408-35-5), sodium polyoxyethylene tridecyl sulfate (25446-78-0), sodium stearate (822-16-2), sodium stearyl sulfate (1120-04-3), sodium toluenesulfonate (12068-03-0), Sodium tridecylbenzene sulfonate (26248-24-8), sodium undecylbenzene sulfonate (27636-75-5), sodium xylene sulfonate (1300-72-7), sorbitan cocoate (68154-36-9), sorbitan monolaurate (1338-39-2), sorbitan monooleate (1338-43-8), sorbitan monopalmitate (26266-57-9), sorbitan monostearate (1338-41-6), sorbitan oleate decylglucoside crosspolymer (1443994-56-6), polymers; sorbitan sesquioleate (8007-43-0),Sorbitan trioleate (26266-58-0), sorbitan, monododecanoate, poly(oxy-1,2-ethanediyl) derivs. (9005-64-5), half sorbitan, monohexadecanoate, poly(oxy-1,2-ethanediyl) derivs. (9005-66-7), sorbitan, monooctadecanoate, poly(oxy-1,2-ethanediyl) derivs. (9005-67-8), half sorbitan, trioctadecanoate, poly(oxy-1,2-ethanediyl) derivs. (9005-71-4), soybean oil fatty acids (68308-53-2), soybean oil, sulfated, sodium salt (61790-16-7), stearic acid (57-11-4), sulfonic acids, C10-18-alkane, sodium salts (68037-49-0), sulfonic acids, C14-16-alkane hydroxy and C14-16-alkene, sodium salts (68439-57-6), sulfonic acids, petroleum, sodium salts (68608-26-4), sulfuric acid, mono-C10-16-alkyl esters, ammonium salts (68081-96-9), sulfuric acid, mono-C10-16-alkyl esters, sodium salts (68585-47-7), sulfuric acid, mono-C12-14 alkyl esters, sodium salts (85586-07-8), sulfuric acid, mono-C12-15-alkyl esters, sodium salts (68890-70-0), sulfuric acid, mono-C12-18-alkyl esters, sodium salts (68955-19-1), sulfuric acid, mono-C14-18-alkyl esters, sodium salts (68081-98-1), sulfuric acid, mono-C16-18-alkyl esters, sodium salts (68955-20-4), sulfuric acid, monococo alkyl esters, sodium salts (97375-27-4), sulfuric acid, monodecyl ester, sodium salt (1:1) (142-87-0), sunflower oil, potassium salt (69669-39-2), tetradecanoic acid (544-63-8), undecyl-D-glucoside (98283-67-1); simethicone and PFAS.

E. Prep B, Microbial Suspensions, and Combinations

Disclosed embodiments concern a concentrated, stabilized and preserved microbial suspension composition. These concentrated, stabilized and preserved microbial suspension compositions are typically formed by combining an effective amount of a microbial suspension composition, referred to herein as a Prep A composition, with an effective amount of a preservative composition, referred to herein as Prep B composition. The Prep B composition can be formed well in advance of being used, such as at least a period of several months. Prep A compositions comprising unpreserved microbial compositions generally should be formed preferably substantially immediately prior to use. Concentrated, stabilized and preserved microbial suspension compositions may be used at the concentration provided by the initial concentrate formulation. Alternatively and more typically, the concentrate may be diluted by an end user to form a ready-to-use composition as desired for an end use application. The amount of dilution can best be determined by the particular end-use application. But, as a general rule, the amount of dilution can be at least substantially equal to the degree to which compositions according to the present invention are concentrated. For example, a 500× concentrate may be diluted up to 1/500× for an end-use application; a 250× concentrate may be diluted up to 1/250× for an end-use application; etc. Dilutions also can be determined as a percentage of the concentrate concentration, and can vary from greater than 0% to 100% dilution, but more typically are equal to ±30%. For example, a 250× concentrate may be diluted by more than 1/250×, such as being diluted within the dilution range of from 1/175 to 1/325, which is equal to ±30% relative to the initial concentrate concentration. Other initial concentrate formulations, such as 80× and 100×, can be diluted in a similar manner, such as to provide ±30% dilutions relative to the concentrate concentration.

Exemplary Prep B formulations are provided below in Table 1.

TABLE 1

| | Prep B - 1 | | Prep B - 2 | |
|---|---|---|---|---|
| Component | (in grams) | | (in grams) | |
| Proxel GXL | 10 | (28.5 wt %) | 13.2 | (28.5 wt %) |
| (15%-20% aqueous dipropylene glycol solution | | | | |
| comprising 1,2-benzoisothiazolin-3-one) | | | | |
| Stepenquat 65 NF | 5 | (14.25 wt %) | 6.6 | (14.25 wt %) |
| (4%-5% ethyl alcohol and 47%-53% C₈₋₁₈ | | | | |
| n-alkyl dimethyl benzyl ammonium chloride) | | | | |
| Propylparaben | 5 | (14.25 wt %) | 6.6 | (14.25 wt %) |
| Propylene Glycol (sterile) | 15 | (42.8 wt %) | 19.8 | (42.7 wt %) |

| | | |
|---|---|---|
| Total | 35 | 46.3 |

Prep B formulations were added to Prep A microbial suspension formulations. These microbial suspensions included, for example, a combination of Bacillus bacteria, namely B. subtilis, B. licheniformis, B. amyloliquefaciens, and B. pumilus. Exemplary resulting concentrated, stabilized and preserved microbial suspension compositions according to the present invention are provided below in Tables 2-4.

TABLE 2

| | Lead Bacillus | | | |
|---|---|---|---|---|
| | 250X Concentrate, | Lead Bacillus | Lead Bacillus | |
| | Dechlorinated | 250X Concentrate, | 250X Concentrate, | Lead Bacillus # 2 |
| | Tap water | Tap water | Deionized water | 250X Concentrate |
| Component | (grams) | (grams) | (grams) | Tap Water |
| Bacillus | 1.45 (B. | 1.05 (B. | 0.68 (B. | 6.59 (B. |
| | Subtilis) | Subtilis) | Subtilis) | Subtilis) |
| | [3.95 × 10⁹ CFUs/mL] | | | |
| Bacillus | 3.49 (B. | 1.27 (B. | 0.81 (B. | 7.91 (B. |
| | licheniformis) | licheniformis) | licheniformis) | licheniformis) |
| | [3.95 × 10⁹ CFUs/mL] | | | |
| Bacillus | 3.98 (B. | 1.6 (B. | 1.0 (B. | 9.89 (B. |
| | amyloliquefaciens) | amyloliquefaciens) | amyloliquefaciens) | amyloliquefaciens) |
| | [3.95 × 10⁹ CFUs/mL] | | | |
| Bacillus | 1.73 (B. | 1.27 (B. | 0.79 (B. | 7.91 (B. |
| | Pumilus) | Pumilus) | Pumilus) | Pumilus) |
| | [3.95 × 10⁹ CFUs/mL] | | | |
| Dipotassium | 2.75 | 1.0 | 0.63 | 1.25 |
| phosphate | | | | |
| Monopotassium | 1.65 | 0.60 | 0.38 | 0.75 |
| Phosphate | | | | |
| Water | 27.9 | 8.83 | 5.52 | 6.54 |
| Propylene Glycol | 28.6 | 10.41 | 6.5 | 17.5 |
| Prep B | 38.5 | 14 | 8.75 | 17.6 |
| Total | 110 | 40 | 25 | 50 |

TABLE 3

| | | | | | Lead Bacillus | | | |
|---|---|---|---|---|---|---|---|---|
| | Lead No. 3 Bacillus | | Lead Bacillus # 4 | | 40X Concentrate, | | Lead Bacillus | |
| | 250X Concentrate | | 250X Concentrate, | | Tap Water | | 80X Concentrate | |
| Component | Tap Water | | Tap Water | | (grams) | | Tap Water | |
| Bacillus | 1.32 grams | | 1.05 mL | | 0.70 mL | | 1.39 mL | |
| | (B. Subtilis) | | (B. Subtilis) | | (B. Subtilis) | | (B. Subtilis) | |
| | [3.95 × 10⁹ CFUs/mL] | | [3.95 × 10⁹ CFUs/mL] | | [6.33 × 10⁸ CFUs/mL] | | [1.27 × 10⁹ CFUs/mL] | |
| Bacillus | 1.58 grams | | 1.27 mL | | 0.56 | | 1.11 mL | |
| | (B. licheniformis) | | (B. licheniformis) | | (B. licheniformis) | | (B. licheniformis) | |
| | [3.95 × 10⁹ CFUs/mL] | | [3.95 × 10⁹ CFUs/mL] | | [6.33 × 10⁸ CFUs/mL] | | | |
| Bacillus | 1.98 grams | | 1.58 mL | | 0.7 mL | | 1.39 mL | |
| | (B. amyloliquefaciens) | | (B. amyloliquefaciens) | | (B. amyloliquefaciens) | | (B. amyloliquefaciens) | |
| | [3.95 × 10⁹ CFUs/mL] | | [3.95 × 10⁹ CFUs/mL] | | [6.33 × 10⁸ CFUs/mL] | | [1.27 × 10⁹ CFUs/mL] | |
| Bacillus | 1.58 grams | | 1.27 mL | | 0.27 | | 0.55 mL | |
| | (B. Pumilus) | | (B. Pumilus) | | (B. Pumilus) | | (B. Pumilus) | |
| | [3.95 × 10⁹ CFUs/mL] | | [3.95 × 10⁹ CFUs/mL] | | [6.33 × 10⁸ CFUs/mL] | | [1.27 × 10⁹ CFUs/mL] | |
| Dipotassium | 1.25 | grams | 1.0 | gram | 0.44 | gram | 0.88 | gram |
| phosphate | | | | | | | | |
| Monopotassium | 0.75 | gram | 0.60 | gram | 0.26 | gram | 0.53 | gram |
| Phosphate | | | | | | | | |
| Water | 6.54 | grams | 8.83 | grams | 58.46 | grams | 52.01 | grams |
| Propylene Glycol | 17.5 | grams | 10.41 | grams | 42.46 | grams | 39.82 | grams |
| Prep B | 17.5 | grams | 14 | grams | 6.16 | grams | 12.32 | grams |

| | | | | |
|---|---|---|---|---|
| Total | 110 | 40 | 110 | 110 |

TABLE 4

| | | | | | Lead Bacillus | |
|---|---|---|---|---|---|---|
| | | | | | 250X Concentrate, | |
| | Lead Bacillus | | Direct to RTU | | 0.1% Tween 80 | |
| | 100X Concentrate | | Lead Bacillus | | Tap Water | |
| Component | Tap Water | | 250X Equivalent | | (grams) | |
| Bacillus | 1.74 mL | | 0.16 mL | | 2.64 mL | |
| | (B. Subtilis) | | (B. Subtilis) | | (B. Subtilis) | |
| | [1.58 × 10⁹ CFUs/mL] | | [1.58 × 10⁷ CFUs/mL] | | [3.95 × 10⁹ CFUs/mL] | |
| Bacillus | 1.39 mL | | 0.13 mL | | 3.16 mL | |
| | (B. licheniformis) | | (B. licheniformis) | | (B. licheniformis) | |
| | [1.58 × 10⁹ CFUs/mL] | | [1.58 × 10⁷ CFUs/mL] | | 3.95 × 10⁹ CFUs/mL] | |
| Bacillus | 1.74 grams | | 0.16 mL | | 3.95 mL | |
| | (B. amyloliquefaciens) | | (B. amyloliquefaciens) | | (B. amyloliquefaciens) | |
| | [1.58 × 10⁹ CFUs/mL] | | [1.58 × 10⁷ CFUs/mL] | | 3.95 × 10⁹ CFUs/mL] | |
| Bacillus | 0.68 mL | | 0.06 mL | | 1.55 mL | |
| | (B. Pumilus) | | (B. Pumilus) | | (B. Pumilus) | |
| | [1.58 × 10⁹ CFUs/mL] | | [1.58 × 10⁷ CFUs/mL] | | 3.95 × 10⁹ CFUs/mL] | |
| Dipotassium | 1.10 | grams | 0.1 | gram | 2.50 | grams |
| phosphate | | | | | | |
| Monopotassium | 0.66 | gram | 0.06 | gram | 1.50 | grams |
| Phosphate | | | | | | |
| Water | 48.79 | grams | 997 | grams | 23.6 | grams |
| Propylene Glycol | 38.5 | grams | 1.57 | grams | 26 | grams |
| Prep B | 15.4 | grams | 2.4 | grams | 35 | grams |
| Tween 80 | | | | | 0.1 | gram |

| | | | |
|---|---|---|---|
| Total | 110 | 1,000 | 100 |

### III. Method for Making

Preparation of concentrated compositions according to the present invention may involve several work-up steps to compensate for narrow solubilization windows. By way of example and without limitation, this may be completed by producing an aqueous bacterial stock (Prep A) and a glycol-based preservation stock (Prep B). Prep B comprises the preservatives along with a portion of the humectant/solvent. Prep A typically is prepared by dissolving a buffering system, such as buffering salts, in minimal water, followed by suspension of the bacterial spores in the aqueous buffer, followed by titration with a fraction of the humectant/solvent. Prep A should be considered un-preserved and typically is made just prior to adding Prep B. Prep B can be made far in advance (at least months), assuming proper homogenization is completed before use. Prep B should be added to Prep A slowly with stirring, and minimal heating as possible to achieve solubility can be used if precipitates appear. Prep B should be added to Prep A at a concentration sufficient for preservation. With reference to amount ranges, solubility of composition components is a primary factor for both the concentrate and diluted product.

### IV. Examples

The following examples are provided to illustrate particular features of certain exemplary embodiments according to the present invention. A person of ordinary skill in the art will understand that the scope of the present invention is not limited to these particular exemplary features.

### Example 1

Microbial suspensions, Prep B formulations, and concentrated, sterilized preservative compositions comprising the microbial suspensions and Prep formulation were made as described above. More particularly, for this example, the microbial suspension is provided below in Table 5, the Prep B formulation is provided in Table 6, and two concentrated, sterilized preservative compositions comprising the microbial suspensions and Prep formulation are provided in Table 7.

TABLE 5
Microbial Suspension

| | | |
|---|---|---|
| Component | Amount | Wt. % |

| | | | |
|---|---|---|---|
| 4 Strain Bacillus | 5.27 | grams | 21.95% |
| Dipotassium phosphate | 1 | gram | 4.17% |
| Monopotassium Phosphate | 0.6 | gram | 2.5% |
| DI Water | 8.8 | mL | 36.7% |
| Sterile Propylene Glycol | 8.4 | grams | 35% |
| Total | 24 | grams | 100% |

TABLE 6
Prep B

| | | |
|---|---|---|
| Component | Amount | Wt. % |

| | | | |
|---|---|---|---|
| Proxel | 9.99 | grams | 28.54% |
| Stepanquat | 5.00 | grams | 14.29% |
| Propylparaben | 5.03 | grams | 14.37% |
| Propylene Glycol | 15.04 | grams | 42.97% |
| Total | 35 | grams | 100% |

TABLE 7
Concentrated, Sterilized Preservative Compositions

| Component | 7A Formulation | 7B Formulation |
|---|---|---|
| Microbial Suspension | 8.41 grams | 8.42 |
| | (60%) | (59.92%) |
| Prep B | 4.90 grams | 4.90 |
| | (34.97%) | (34.88%) |
| Propylene Glycol | 0.70 gram | 0.73 |
| | (5%) | (5.2%) |
| Total | 14.01 grams | 14.05 grams |

The 7A and 7B formulations were then used to form dilutions periodically over time and the diluted compositions were assayed for the number of CFUs present under three different conditions: 1. Using Prep B immediately after forming the composition; 2. Using Prep B 3 days after its initial formulation; 3. Microbial suspension compositions were stored at 42° C. T The results of these tests are provided below in Tables 8-13. Dilution values reported in the following tables have been rounded to a single significant figure.

TABLE 8
CFU Counts

| Formulation | Days | Dilution | Counts | Average Counts | CFU/mL |
|---|---|---|---|---|---|
| 250x 7A | 0 | 8 | (96, 106, 106, 95, 125) | 105.6 | 1.06E+10 |
| 250x 7A | 3 | 8 | (11, 7, 5, 12, 19) | 10.8 | 1.07E+10 |
| 250x 7A | 4 | 9 | (146, 175) | 160.5 | 1.60E+10 |
| 250x 7A | 35 | 9 | (193, 196, 215, 191, 194) | 197.8 | 1.97E+10 |
| 250x 7A | 88 | 9 | (178, 150, 168, 180, 143) | 163.8 | 1.63E+10 |
| 250x 7A | 217 | 9 | (199, 180, 182, 263, 195) | 203.8 | 2.03E+10 |
| 250x 7A | 305 | 9 | (71, 77, 146, 99, 98) | 98.2 | 9.78E+09 |
| 250x 7A | 326 | 9 | (80, 100, 94, 77, 97) | 89.6 | 8.92E+09 |
| 250x 7A | 391 | 9 | (130, 132, 143, 162) | 141.8 | 1.41E+10 |
| 250x 7A | 606 | 9 | (211, 230, 226, 255, 253) | 235 | 2.34E+10 |

TABLE 9
CFU Counts

| Formulation | Days | Dilution | Counts | Average Counts | CFU/mL |
|---|---|---|---|---|---|
| 250x 7A - Prepped on | 0 | 6 | (53, 40, 46, 47, 55) | 48.2 | 4.82E+07 |
| T + 3 (of 250X) | | | | | |
| 250x 7A - Prepped on | 32 | 6 | (50, 60, 47, 48, 62) | 53.4 | 5.34E+07 |
| T + 3 (of 250X) | | | | | |
| 250x 7A - Prepped on | 49 | 6 | (67, 53, 45, 68) | 58.25 | 5.83E+07 |
| T + 3 (of 250X) | | | | | |
| 250x 7A - Prepped on | 70 | 6 | (47, 44, 51, 44, 51) | 47.4 | 4.74E+07 |
| T + 3 (of 250X) | | | | | |
| 250x 7A - Prepped on | 85 | 6 | (52, 48, 52, 69, 66) | 57.4 | 5.74E+07 |
| T + 3 (of 250X) | | | | | |
| 250x 7A - Prepped on | 134 | 6 | (53, 49, 46) | 49.3333 | 4.93E+07 |
| T + 3 (of 250X) | | | | | |
| 250x 7A - Prepped on | 170 | 6 | (52, 51, 42, 52, 49) | 49.2 | 4.92E+07 |
| T + 3 (of 250X) | | | | | |
| 250x 7A - Prepped on | 198 | 6 | (63, 63, 50, 51, 52) | 55.8 | 5.58E+07 |
| T + 3 (of 250X) | | | | | |
| 250x 7A - Prepped on | 214 | 6 | (56, 47, 46, 44, 49) | 48.4 | 4.84E+07 |
| T + 3 (of 250X) | | | | | |
| 250x 7A - Prepped on | 302 | 6 | (4, 6, 5, 13) | 7 | 7.00E+06 |
| T + 3 (of 250X) | | | | | |
| 250x 7A - Prepped on | 323 | 6 | (54, 47, 55, 55) | 52.75 | 5.28E+07 |
| T + 3 (of 250X) | | | | | |
| 250x 7A - Prepped on | 388 | 6 | (46, 48, 54, 61, 62) | 54.2 | 5.42E+07 |
| T + 3 (of 250X) | | | | | |
| 250x 7A - Prepped on | 595 | 6 | (52, 47, 57, 61, 36) | 50.6 | 5.06E+07 |
| T + 3 (of 250X) | | | | | |
| 250x 7A - Prepped on | 591 | 6 | (45, 31, 38, 41, 45) | 40 | 4.00E+07 |
| T + 3 (of 250X) | | | | | |
| 250x 7A - Prepped on | 599 | 6 | (33, 32, 24, 37, 28) | 30.8 | 3.08E+07 |
| T + 3 (of 250X) | | | | | |
| 250x 7A - Prepped on | 599 | 6 | (45, 32, 37, 33, 40) | 37.4 | 3.74E+07 |
| T + 3 (of 250X) | | | | | |
| 250x 7A - Prepped on | 605 | 6 | (39, 35, 32, 36, 36) | 35.6 | 3.56E+07 |
| T + 3 (of 250X) | | | | | |

TABLE 10
CFU Counts

| Formulation | Days | Dilution | Counts | Average Counts | CFU/mL |
|---|---|---|---|---|---|
| 1/250x 7A 42° C. | 302 | 6 | (6, 1, 4, 2, 4) | 3.4 | 3.40E+06 |
| 1/250x 7A 42° C. | 323 | 6 | (10, 18, 20, 7, 12) | 13.4 | 1.34E+07 |
| 1/250x 7A 42° C. | 388 | 6 | (30, 31, 32, 38, 55) | 37.2 | 3.72E+07 |
| 1/250x 7A 42° C. | 595 | 6 | (6, 7, 4, 5, 2) | 4.8 | 4.80E+06 |

TABLE 11
CFU Counts

| Formulation | Days | Dilution | Counts | Average Counts | CFU/mL |
|---|---|---|---|---|---|
| 250x 7B | 0 | 8 | (87, 95, 98, 107, 96) | 96.6 | 9.66E+09 |
| 250x 7B | 3 | 8 | (21, 12, 20, 20, 19) | 18.4 | 1.82E+10 |
| 250x 7B | 4 | 9 | 132 | 132 | 1.31E+10 |
| 250x 7B | 35 | 9 | (178, 206, 212, 218) | 203.5 | 2.03E+10 |
| 250x 7B | 88 | 9 | (198, 230, 170, 175) | 193.25 | 1.92E+10 |
| 250x 7B | 217 | 9 | (129, 173, 170, 163) | 158.75 | 1.58E+10 |
| 250x 7B | 305 | 9 | (89, 92, 72, 87, 87) | 85.4 | 8.51E+09 |
| 250x 7B | 391 | 9 | (125, 128, 138, 152, 153) | 139.2 | 1.39E+10 |
| 250x 7B | 606 | 9 | (195, 177, 190, 190, 180) | 186.4 | 1.86E+10 |

TABLE 12
CFU Counts

| Formulation | Days | Dilution | Counts | Average Counts | CFU/mL |
|---|---|---|---|---|---|
| 1/250x 7B - Prepped on | 0 | 6 | (50, 64, 43, 48) | 51.25 | 5.13E+07 |
| T + 3 (of 250X) | | | | | |
| 1/250x 7B - Prepped on | 32 | 6 | (48, 52, 62, 52) | 53.5 | 5.35E+07 |
| T + 3 (of 250X) | | | | | |
| 1/250x 7B - Prepped on | 49 | 6 | (113, 118, 115, 114, 112) | 114.4 | 1.14E+08 |
| T + 3 (of 250X) | | | | | |
| 1/250x 7B - Prepped on | 50 | 6 | (88, 71, 83, 99, 81) | 84.4 | 8.44E+07 |
| T + 3 (of 250X) | | | | | |
| 1/250x 7B - Prepped on | 70 | 6 | (64, 81, 72, 74) | 72.75 | 7.28E+07 |
| T + 3 (of 250X) | | | | | |
| 1/250x 7B - Prepped on | 85 | 6 | (74, 76, 77, 79, 79) | 77 | 7.70E+07 |
| T + 3 (of 250X) | | | | | |
| 1/250x 7B - Prepped on | 134 | 6 | (79, 69, 101) | 83 | 8.30E+07 |
| T + 3 (of 250X) | | | | | |
| 1/250x 7B - Prepped on | 170 | 6 | (57, 61, 72, 50, 65) | 61 | 6.10E+07 |
| T + 3 (of 250X) | | | | | |
| 1/250x 7B - Prepped on | 198 | 6 | (68, 77, 78, 67, 75) | 73 | 7.30E+07 |
| T + 3 (of 250X) | | | | | |
| 1/250x 7B - Prepped on | 214 | 6 | (36, 43, 44, 82) | 51.25 | 5.13E+07 |
| T + 3 (of 250X) | | | | | |
| 1/250x 7B - Prepped on | 305 | 6 | (40, 30, 157, 42) | 67.25 | 6.73E+07 |
| T + 3 (of 250X) | | | | | |
| 1/250x 7B - Prepped on | 388 | 6 | (21, 28, 37, 40, 44) | 34 | 3.40E+07 |
| T + 3 (of 250X) | | | | | |
| 1/250x 7B - Prepped on | 389 | 6 | (34, 32, 36, 44, 21) | 33.4 | 3.34E+07 |
| T + 3 (of 250X) | | | | | |
| 1/250x 7B - Prepped on | 407 | 6 | (19, 18, 23, 19, 16) | 19 | 1.90E+07 |
| T + 3 (of 250X) | | | | | |
| 1/250x 7B - Prepped on | 409 | 6 | (49, 55, 61, 67, 54) | 57.2 | 5.72E+07 |
| T + 3 (of 250X) | | | | | |
| 1/250x 7B - Prepped on | 438 | 6 | (20, 26, 24, 27, 16) | 22.6 | 2.26E+07 |
| T + 3 (of 250X) | | | | | |
| 1/250x 7B - Prepped on | 441 | 6 | (39, 50, 42, 40, 47) | 43.6 | 4.36E+07 |
| T + 3 (of 250X) | | | | | |
| 1/250x 7B - Prepped on | 591 | 6 | (17, 21, 13, 17, 19) | 17.4 | 1.74E+07 |
| T + 3 (of 250X) | | | | | |
| 1/250x 7B - Prepped on | 595 | 6 | (11, 3, 6, 10, 11) | 8.2 | 8.20E+06 |
| T + 3 (of 250X) | | | | | |
| 1/250x 7B - Prepped on | 595 | 6 | (14, 11, 8, 8, 14) | 11 | 1.10E+07 |
| T + 3 (of 250X) | | | | | |
| 1/250x 7B - Prepped on | 596 | 6 | (13, 7, 7, 8, 8) | 8.6 | 8.60E+06 |
| T + 3 (of 250X) | | | | | |
| 1/250x 7B - Prepped on | 596 | 6 | (2, 3, 1, 1, 1) | 1.6 | 1.60E+06 |
| T + 3 (of 250X) | | | | | |
| 1/250x 7B - Prepped on | 598 | 6 | (4, 3, 3, 4, 2) | 3.2 | 3.20E+06 |
| T + 3 (of 250X) | | | | | |
| 1/250x 7B - Prepped on | 598 | 6 | (5, 10, 9, 4, 6) | 6.8 | 6.80E+06 |
| T + 3 (of 250X) | | | | | |
| 1/250x 7B - Prepped on | 605 | 5 | (41, 48, 42, 40, 32) | 40.6 | 4.06E+06 |
| T + 3 (of 250X) | | | | | |

TABLE 13
CFU Counts

| Formulation | Days | Dilution | Counts | Average Counts | CFU/mL |
|---|---|---|---|---|---|
| 1/250x 7B 42 C. | 305 | 6 | (91, 167, 91, 161, 119) | 125.8 | 1.26E+08 |
| 1/250x 7B 42 C. | 388 | 6 | (4, 11, 12, 14, 17) | 11.6 | 1.16E+07 |
| 1/250x 7B 42 C. | 595 | 6 | (7, 11, 8, 14, 7) | 9.4 | 9.40E+06 |

These CFU assays establish that the target Bacillus spores do not die-off by the inherent action of the preservative system.

The formulations of this example were also used in Gram negative bacterial and yeast challenges. Challenge tests were generally conducted as follows:

Gram negative (G-) organisms were always represented by two Pseudomonads. Stock compositions were made by reconstituting 1 loop (an inoculation loop, i.e. a small, e.g. ˜5 mm, or enough for ˜10 uL of water, metal wire looped for picking up microbial colonies off of an agar plate) of each strain into 9.9 mL sterile dilution buffer. The resulting suspension varied in concentration, but was typically within one log of previous/later stocks.

Mold organisms were initially represented by a combination of multiple (typically 5+) mold strains pulled as a unitary conglomerate from the surface of a PDA plate, and reconstituted in 9.9 mL sterile dilution buffer. This proved highly variable and difficult to control, but eventually a slower-growing species was identified to represent the mold category (in photos). Even with this particular strain isolated, mold challenges tended to generate an oppressively high proportion of invalid results. This, along with the perceived redundancy of running both yeast and mold in high throughput screening, resulted in removing mold from follow-up testing.

Yeast organisms were represented by a particular unidentified environmental yeast found growing in partially preserved liquid products. They were handled in a similar fashion as G−.

9 mL of test formulation (or sterile dilution buffer for control) was spiked with 1 mL respective challenge stock and struck on selective agar, respective to insult type. Samples were incubated and restruck at daily intervals through 3 additional days, where two observations were made: (1) presence/absence of culture; and (2) comparison to a control quadrant (photographic). A + or − indicates presence or absence, respectively, while in later cases we started reporting +/− and −/+ to represent varying degrees of culture thinning compared to control (+/− was mild thinning, −/+ was nearly devoid of insult organism). Any reduction versus the control was seen as a positive response, since the implication is insult die-off or lessened growth. Lastly, + or − results were aligned by T+ days; T+0 was never expected to have full negative results, since the goal is not necessarily antiseptic activity. Results progressing toward negative by T+3 were considered indicators of successful preservation, assuming negative controls remained positive.

Product iterations were sampled after many months, often with truncated/endpoint-only testing—i.e. running until negative. An indicator of long-term preservative stability would be continued successful negatives or reductions any time after T+0. Mold was dropped from stability testing in these cases.

The challenge method, while somewhat crude to improve throughput, did not employ defined insult concentrations; nevertheless, the insults were always quite strong (i.e. higher than would ever realistically be seen in the field), as well as including other limit-based parameters (e.g. the 90% inclusion rate favors growth), so the qualitative purpose was ultimately served.

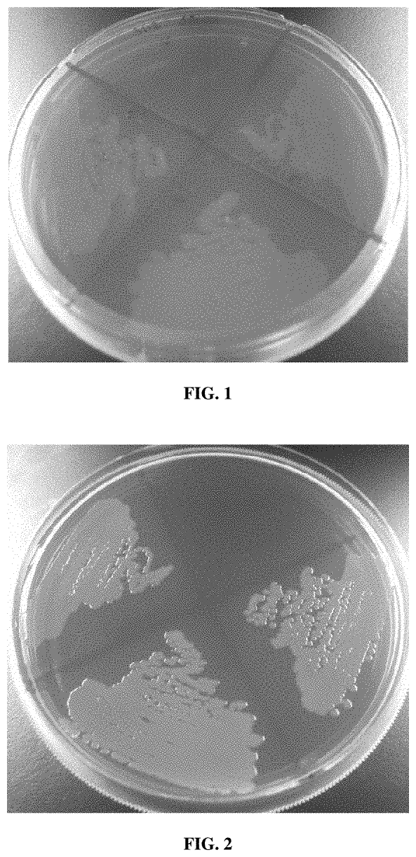

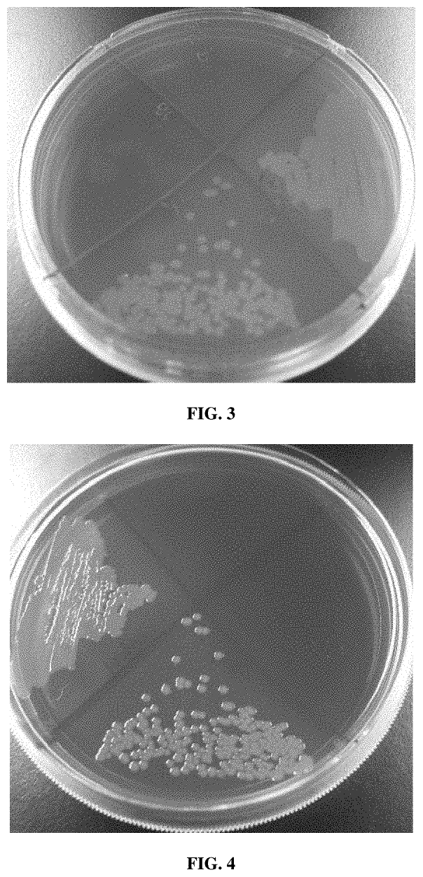

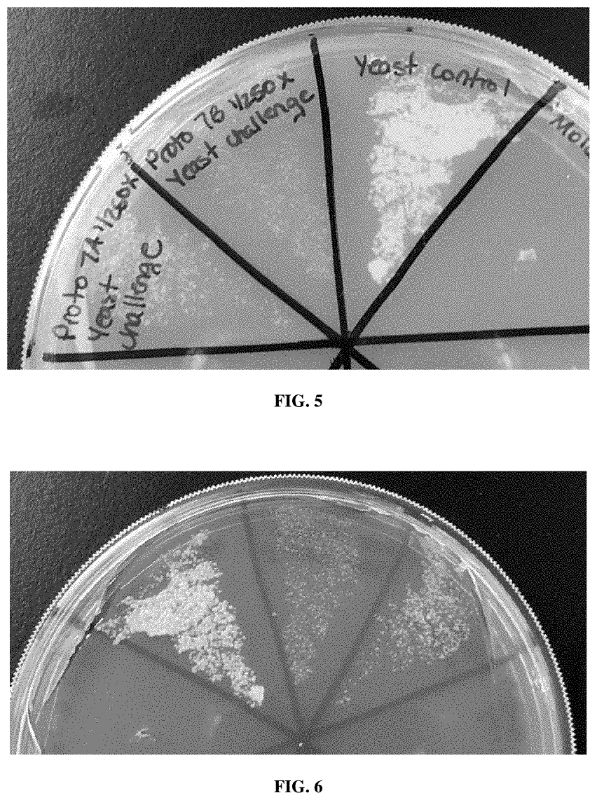

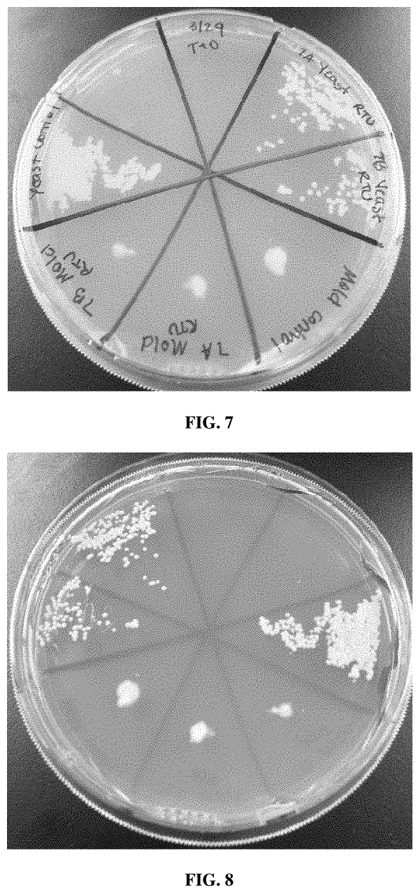

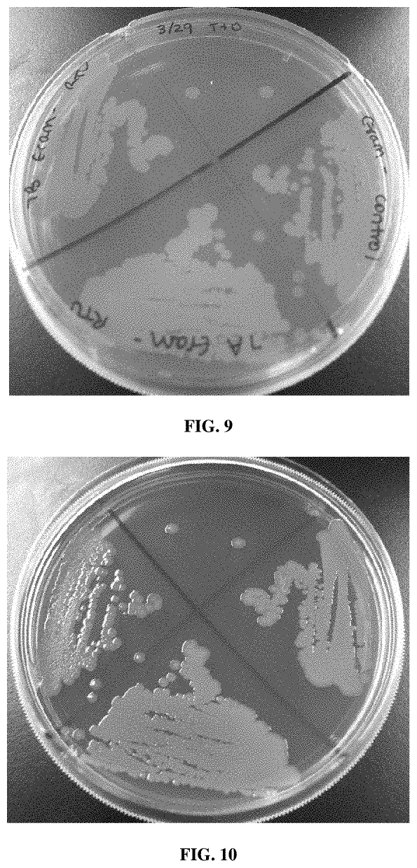

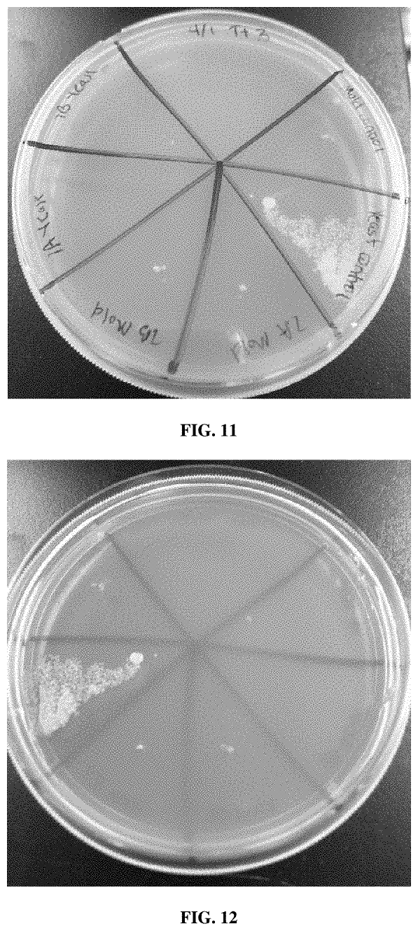

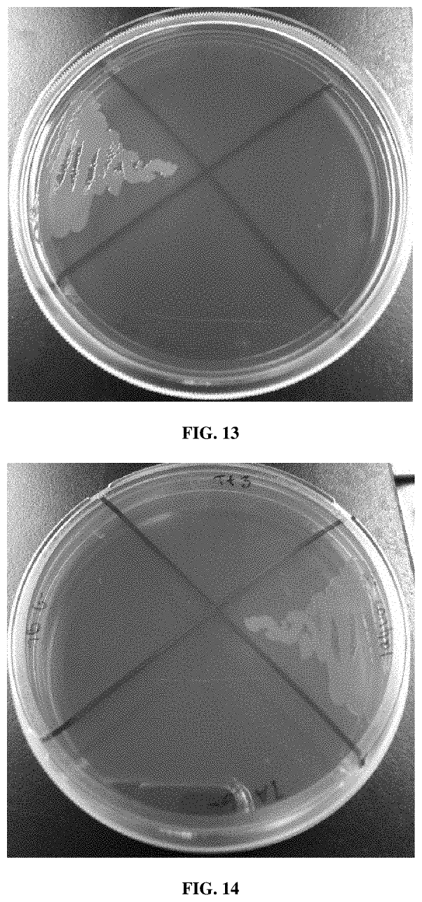

The results of the challenge studies for this example are illustrated by FIGS. 1-14, which clearly establish that compositions according to the present invention had substantially reduced yeast and mold contamination relative to a control composition that did not include a Prep B preservative composition according to the present invention.

### Example 2

Microbial suspensions, Prep B formulations, and concentrated, sterilized preservative compositions comprising the microbial suspensions and Prep formulation were made as described above. More particularly, for this example, concentrated, sterilized preservative compositions comprising the microbial suspensions and Prep B formulation are provided above in Tables 2-4. These formulations were then used to form dilutions periodically over time. The diluted compositions were then challenge with bacteria, yeast, mold, or combinations thereof, and then assayed for the number of CFUs present. The results of these tests are provided below in Tables 14-67.

TABLE 14
CFU Counts

| T(days) | Sample | Dilution | Counts | Average | CFU/mL |
|---|---|---|---|---|---|
| 0 | Lead Bacillus 250X, | 8 | (62, 65, 64, 58, 71) | 64.00 | 6.40E+09 |
| | Dechlorinated Tap Water | | | | |
| 1 | Lead Bacillus 250X, | 8 | (56, 70, 67, 95, 72) | 72.00 | 7.20E+09 |
| | Dechlorinated Tap Water | | | | |
| 8 | Lead Bacillus 250X, | 8 | (47, 53, 52, 62) | 53.50 | 5.35E+09 |
| | Dechlorinated Tap Water | | | | |
| 10 | Lead Bacillus 250X, | 8 | (107, 103, 103, 101, 114) | 105.60 | 1.06E+10 |
| | Dechlorinated Tap Water | | | | |
| 14 | Lead Bacillus 250X, | 8 | (44, 50, 68, 61) | 55.75 | 5.58E+09 |
| | Dechlorinated Tap Water | | | | |
| 14 | Lead Bacillus 250X, | 8 | (70, 82, 65, 70, 70) | 71.40 | 7.14E+09 |
| | Dechlorinated Tap Water | | | | |
| 14 | Lead Bacillus 250X, | 8 | (68, 76, 60, 88, 81) | 74.60 | 7.46E+09 |
| | Dechlorinated Tap Water | | | | |
| 85 | Lead Bacillus 250X, | 8 | (105, 39, 24, 34) | 50.50 | 5.05E+09 |
| | Dechlorinated Tap Water | | | | |
| 185 | Lead Bacillus 250X, | 8 | (93, 121, 103, 77) | 98.50 | 9.85E+09 |
| | Dechlorinated Tap Water | | | | |
| 372 | Lead Bacillus 250X, | 8 | (100, 79, 74, 88, 80) | 84.20 | 8.42E+09 |
| | Dechlorinated Tap Water | | | | |
| 543 | Lead Bacillus 250X, | 8 | (59, 51, 52, 62) | 56.75 | 5.68E+09 |
| | Dechlorinated Tap Water | | | | |

TABLE 15
CFU Counts

| T(days) | Sample | Dilution | Counts | Average | CFU/mL |
|---|---|---|---|---|---|
| 15 | Lead Bacillus 250X, | 8 | (26, 31, 47, 39, 34) | 35.40 | 3.54E+09 |
| | Dechlorinated Tap Water 42° C. | | | | |
| 29 | Lead Bacillus 250X, | 8 | (33, 50, 52, 33) | 42.00 | 4.20E+09 |
| | Dechlorinated Tap Water 42° C. | | | | |
| 75 | Lead Bacillus 250X, | 8 | (67, 55, 41, 49, 39) | 50.20 | |
| | Dechlorinated Tap Water 42° C. | | | | |
| 96 | Lead Bacillus 250X, | 8 | (37, 34, 37, 23, 28) | 31.80 | 3.18E+09 |
| | Dechlorinated Tap Water 42° C. | | | | |
| 85 | Lead Bacillus 250X, | 8 | (30, 27, 34, 31, 30) | 30.40 | 3.04E+09 |
| | Dechlorinated Tap Water 42° C. | | | | |
| 358 | Lead Bacillus 250X, | 8 | (35, 52, 58) | 48.33 | 4.83E+09 |
| | Dechlorinated Tap Water 42° C. | | | | |

TABLE 16
CFU Counts

| T(days) | Sample | Dilution | Counts | Average | CFU/mL |
|---|---|---|---|---|---|
| 0 | Lead Bacillus 1/250X, | 6 | (46, 34, 43, 42, 46) | 42.20 | 4.22E+07 |
| | Dechlorinated Tap Water | | | | |
| 1 | Lead Bacillus 1/250X, | 6 | (48, 75, 62, 66) | 62.75 | 6.28E+07 |
| | Dechlorinated Tap Water | | | | |
| 8 | Lead Bacillus 1/250X, | 6 | 64 | 64.00 | 6.40E+07 |
| | Dechlorinated Tap Water | | | | |
| 10 | Lead Bacillus 1/250X, | 6 | (93, 173, 96, 142, 104) | 121.60 | 1.22E+08 |
| | Dechlorinated Tap Water | | | | |
| 29 | Lead Bacillus 1/250X, | 6 | — | — | — |
| | Dechlorinated Tap Water | | | | |
| 30 | Lead Bacillus 1/250X, | 6 | (20, 26, 37, 23, 27) | 26.60 | 2.66E+07 |
| | Dechlorinated Tap Water | | | | |
| 65 | Lead Bacillus 1/250X, | 6 | (26, 22, 34, 19, 17) | 23.60 | 2.36E+07 |
| | Dechlorinated Tap Water | | | | |
| 85 | Lead Bacillus 1/250X, | 6 | (56, 37, 109, 56) | 64.50 | 6.45E+07 |
| | Dechlorinated Tap Water | | | | |
| 184 | Lead Bacillus 1/250X, | 6 | (12, 26, 15, 17, 10) | 16.00 | 1.60E+07 |
| | Dechlorinated Tap Water | | | | |
| 190 | Lead Bacillus 1/250X, | 6 | (7, 11, 8, 12, 27) | 13.00 | 1.30E+07 |
| | Dechlorinated Tap Water | | | | |
| 192 | Lead Bacillus 1/250X, | 6 | (22, 17, 18, 22, 13) | 18.40 | 1.84E+07 |
| | Dechlorinated Tap Water | | | | |
| 372 | Lead Bacillus 1/250X, | 6 | (7, 9, 6, 7, 14) | 8.60 | 8.60E+06 |
| | Dechlorinated Tap Water | | | | |
| 393 | Lead Bacillus 1/250X, | 6 | (2, 3, 2, 4, 2) | 2.60 | 2.60E+06 |
| | Dechlorinated Tap Water | | | | |

TABLE 17
CFU Counts

| T(days) | Sample | Dilution | Counts | Average | CFU/mL |
|---|---|---|---|---|---|
| 0 | Lead Bacillus 1/250X | 6 | (30, 38) | 34.00 | 3.40E+07 |
| | T(stock + 15) | | | | |
| 14 | Lead Bacillus 1/250X | 6 | — | — | — |
| | T(stock + 15) | | | | |
| 15 | Lead Bacillus 1/250X | 6 | (25, 31, 24, 24, 21) | 25.00 | 2.50E+07 |
| | T(stock + 15) | | | | |
| 50 | Lead Bacillus 1/250X | 6 | (24, 11, 23, 20, 18) | 19.20 | 1.92E+07 |
| | T(stock + 15) | | | | |
| 72 | Lead Bacillus 1/250X | 6 | (22, 25, 30, 21) | 24.50 | 2.45E+07 |
| | T(stock + 15) | | | | |
| 169 | Lead Bacillus 1/250X | 6 | (7, 10, 11, 11) | 9.75 | 9.75E+06 |
| | T(stock + 15) | | | | |
| 177 | Lead Bacillus 1/250X | 6 | (19, 24, 31, 26, 24) | 24.80 | 2.48E+07 |
| | T(stock + 15) | | | | |
| 357 | Lead Bacillus 1/250X | 6 | (6, 4, 5, 6, 8) | 5.80 | 5.80E+06 |
| | T(stock + 15) | | | | |
| 360 | Lead Bacillus 1/250X | 5 | (23, 31, 24, 17) | 23.75 | 2.38E+06 |
| | T(stock + 15) | | | | |
| 364 | Lead Bacillus 1/250X | 6 | (0, 3, 0, 1, 1) | 1.00 | 1.00E+06 |
| | T(stock + 15) | | | | |
| 364 | Lead Bacillus 1/250X | 6 | (3, 1, 0, 3) | 1.75 | 1.75E+06 |
| | T(stock + 15) | | | | |
| 364 | Lead Bacillus 1/250X | 6 | (0, 0, 0, 0, 0) | 0.00 | 0.00E+00 |
| | T(stock + 15) | | | | |
| 371 | Lead Bacillus 1/250X | 5 | (38, 40, 43, 41, 47) | 41.80 | 4.18E+06 |
| | T(stock + 15) | | | | |
| 380 | Lead Bacillus 1/250X | 5 | (16, 13, 22, 14, 20) | 17.00 | 1.70E+06 |
| | T(stock + 15) | | | | |
| 387 | Lead Bacillus 1/250X | 5 | (10, 9, 11, 5, 10) | 9.00 | 9.00E+05 |
| | T(stock + 15) | | | | |

TABLE 18
CFU Counts

| T(days) | Sample | Dilution | Counts | Average | CFU/mL |
|---|---|---|---|---|---|
| 0 | Mass Balance Pour-out of | 5 | (4, 5, 5, 2, 2) | 3.60 | 3.60E+05 |
| | Lead #1 Bacillus 1/250x | | | | |
| | T(stock +15) | | | | |
| 64 | Mass Balance Pour-out of | 5 | (1, 11, 2) | 4.67 | 4.67E+05 |
| | Lead #1 Bacillus 1/250x | | | | |
| | T(stock +15) | | | | |

TABLE 19
CFU Counts

| T(days) | Sample | Dilution | Counts | Average | CFU/mL |
|---|---|---|---|---|---|
| 0 | Poured-out, added 116.34 g, | 4 | — | — | — |
| | 0.25% Tween 80, 1% Kathon | | | | |
| | reconstituted | | | | |
| 1 | Poured-out, added 116.34 g, | 5 | (125, 125, 134, 127, 154) | 133.00 | 1.33E+07 |
| | 0.25% Tween 80, 1% Kathon | | | | |
| | reconstituted | | | | |
| 64 | Poured-out, added 116.34 g, | 5 | (66, 112, 67, 51, 63) | 71.80 | 7.18E+06 |
| | 0.25% Tween 80, 1% Kathon | | | | |
| | reconstituted | | | | |

TABLE 20
CFU Counts

| T(days) | Sample | Dilution | Counts | Average | CFU/mL |
|---|---|---|---|---|---|
| 0 | Lead Bacillus 1/250X | 6 | (35, 25, 46, 32, 33) | 34.20 | 3.42E+07 |
| | T(stock + 385) | | | | |
| 4 | Lead Bacillus 1/250X | 6 | (31, 18, 32, 24, 18) | 24.60 | 2.46E+07 |
| | T(stock + 385) | | | | |
| 7 | Lead Bacillus 1/250X | 6 | (27, 39, 25, 33, 29) | 30.60 | 3.06E+07 |
| | T(stock + 385) | | | | |

TABLE 21
CFU Counts

| T(days) | Sample | Dilution | Counts | Average | CFU/mL |
|---|---|---|---|---|---|
| 7 | Lead Bacillus 1/250X | 6 | (26, 17, 25, 29, 28) | 25.00 | 2.48E+07 |
| | T(stock + 385) Window | | | | |

TABLE 22
CFU Counts

| T(days) | Sample | Dilution | Counts | Average | CFU/mL |
|---|---|---|---|---|---|
| 1 | Lead Bacillus 1/250x, | 6 | (19, 22, 22, 19, 16) | 19.60 | 1.96E+07 |
| | Dilution Bottle | | | | |

TABLE 23
CFU Counts

| T(days) | Sample | Dilution | Counts | Average | CFU/mL |
|---|---|---|---|---|---|
| 1 | Lead Bacillus 1/250x 3% | 6 | (30, 35, 39, 33, 40) | 35.40 | 3.54E+07 |
| | Tween 80, Dilution Bottle | | | | |
| 143 | Lead Bacillus 1/250x 3% | 6 | (59, 19, 22, 22) | 30.50 | 3.05E+07 |
| | Tween 80, Dilution Bottle | | | | |

TABLE 24
CFU Counts

| T(days) | Sample | Dilution | Counts | Average | CFU/mL |
|---|---|---|---|---|---|
| 1 | Lead Bacillus 1/250x 0.1 mL | 6 | (54, 81, 50, 39, 30) | 50.80 | 5.08E+07 |
| | Tween 80 added, Dilution Bottle | | | | |

TABLE 25
CFU Counts

| T(days) | Sample | Dilution | Counts | Average | CFU/mL |
|---|---|---|---|---|---|
| 1 | Lead Bacillus 1/250x 1.5% | 6 | (36, 42, 36, 33, 35) | 36.40 | 3.64E+07 |
| | Tween 80, Dilution Bottle | | | | |

TABLE 26
CFU Counts

| T(days) | Sample | Dilution | Counts | Average | CFU/mL |
|---|---|---|---|---|---|
| 1 | Lead Bacillus 1/250x 0.5% | 6 | (35, 31, 26, 32, 36) | 32.00 | 3.20E+07 |
| | Tween 80, Dilution Bottle | | | | |

TABLE 27
CFU Counts

| T(days) | Sample | Dilution | Counts | Average | CFU/mL |
|---|---|---|---|---|---|
| 1 | Lead Bacillus 1/250x 0.1% | 6 | (33, 24, 32, 29, 26) | 28.80 | 2.88E+07 |
| | Tween 80, Dilution Bottle | | | | |
| 0 | Lead Bacillus 1/250x 0.1% | 6 | (19, 17, 16, 13) | 16.25 | 1.63E+07 |
| | Tween 80, Dilution Bottle | | | | |

TABLE 28
CFU Counts

| T(days) | Sample | Dilution | Counts | Average | CFU/mL |
|---|---|---|---|---|---|
| 0 | 500 uL (30 uL 1:10 Tween 80 + | 6 | (11, 15, 14) | 13.33 | 1.33E+07 |
| | 3 mL Lead 250x Concentrate) + | | | | |
| | 124.5 mL DIW | | | | |
| 0 | 500 uL (30 uL 1:10 Tween 80 + | 6 | (21, 24, 25, 16) | 21.50 | 2.15E+07 |
| | 3 mL Lead 250x Concentrate) + | | | | |
| | 124.5 mL DIW | | | | |

CFU Counts

| T(days) | Sample | Dilution | Counts | Average | CFU/mL |
|---|---|---|---|---|---|
| 1 | Lead Bacillus 1/250x 0.05% | 6 | (22, 19, 23, 23, 24) | 22.20 | 2.22E+07 |
| | Tween 80, Dilution Bottle | | | | |

TABLE 30
CFU Counts

| T(days) | Sample | Dilution | Counts | Average | CFU/mL |
|---|---|---|---|---|---|
| 0 | Lead #2 Bacillus 250X, Tap Water | 8 | (107, 114, 112, 99) | 108.00 | 1.08E+10 |
| 69 | Lead #2 Bacillus 250X, Tap Water | 8 | (79, 86) | 82.50 | 8.25E+09 |
| 169 | Lead #2 Bacillus 250X, Tap Water | 8 | (76, 94, 97, 87, 114) | 93.60 | 9.36E+09 |
| 356 | Lead #2 Bacillus 250X, Tap Water | 8 | (95, 70, 89, 100, 79) | 86.60 | 8.66E+09 |

TABLE 31
CFU Counts

| T(days) | Sample | Dilution | Counts | Average | CFU/mL |
|---|---|---|---|---|---|
| 0 | Lead #2 Bacillus 250X, DI Water | 8 | (89, 77, 90, 76) | 83.00 | 8.30E+09 |
| 69 | Lead #2 Bacillus 250X, DI Water | 8 | (124, 84, 72, 56, 73) | 81.80 | 8.18E+09 |
| 169 | Lead #2 Bacillus 250X, DI Water | 8 | (88, 114, 108, 116, 106) | 106.40 | 1.06E+10 |
| 356 | Lead #2 Bacillus 250X, DI Water | 8 | (95, 97, 117, 110, 108) | 105.40 | 1.05E+10 |

TABLE 32
CFU Counts

| T(days) | Sample | Dilution | Counts | Average | CFU/mL |
|---|---|---|---|---|---|
| 0 | Lead #2 Bacillus 1/250X, Tap Water | 6 | (23, 22, 27, 35, 15) | 24.40 | 2.44E+07 |
| 69 | Lead #2 Bacillus 1/250X, Tap Water | 8 | (66, 71) | 68.50 | 6.85E+09 |
| 168 | Lead #2 Bacillus 1/250X, Tap Water | 6 | (20, 23, 17, 20, 24) | 20.80 | 2.08E+07 |
| 175 | Lead #2 Bacillus 1/250X, Tap Water | 6 | (44, 25, 19, 19, 25) | 26.40 | 2.64E+07 |
| 176 | Lead #2 Bacillus 1/250X, Tap Water | 6 | (56, 52, 53, 50, 27) | 47.60 | 4.76E+07 |
| 356 | Lead #2 Bacillus 1/250X, Tap Water | 6 | (25, 33, 28, 25, 19) | 26.00 | 2.60E+07 |
| 377 | Lead #2 Bacillus 1/250X, Tap Water | 6 | (14, 19, 14, 20, 13) | 16.00 | 1.60E+07 |

TABLE 33
CFU Counts

| T(days) | Sample | Dilution | Counts | Average | CFU/mL |
|---|---|---|---|---|---|
| 0 | Lead #2 Bacillus 1/250X, DI Water | 6 | (28, 39, 34, 33, 37) | 34.20 | 3.42E+07 |
| 48 | Lead #2 Bacillus 1/250X, DI Water | 6 | (17, 12, 11, 8, 13) | 12.20 | 1.22E+07 |
| 69 | Lead #2 Bacillus 1/250X, DI Water | 6 | (21, 56, 31, 16) | 31.00 | 3.10E+07 |
| 168 | Lead #2 Bacillus 1/250X, DI Water | 6 | (10, 8, 10, 6, 4) | 7.60 | 7.60E+06 |
| 176 | Lead #2 Bacillus 1/250X, DI Water | 6 | (16, 19, 17, 14, 10) | 15.20 | 1.52E+07 |
| 356 | Lead #2 Bacillus 1/250X, DI Water | 6 | (0, 5, 1, 0, 1) | 1.40 | 1.40E+06 |
| 357 | Lead #2 Bacillus 1/250X, DI Water | 6 | (2, 2, 3, 2, 4) | 2.60 | 2.60E+06 |
| 377 | Lead #2 Bacillus 1/250X, DI Water | 6 | (0, 0, 3, 2, 1) | 1.20 | 1.20E+06 |

TABLE 34
CFU Counts

| T(days) | Sample | Dilution | Counts | Average | CFU/mL |
|---|---|---|---|---|---|
| 0 | Lead #2 Bacillus 1/250X, DI Water | 6 | (30, 40, 39, 30, 46) | 37.00 | 3.70E+07 |
| | T(stock + 369) | | | | |
| 4 | Lead #2 Bacillus 1/250X, DI Water | 6 | (29, 33, 30, 29, 34) | 31.00 | 3.10E+07 |
| | T(stock + 369) | | | | |

TABLE 35
CFU Counts

| T(days) | Sample | Dilution | Counts | Average | CFU/mL |
|---|---|---|---|---|---|
| 0 | Bacillus | 9 | (84, 59, 74, 56, 67) | 68.00 | 6.80E+10 |
| | Pre-Mix | | | | |

TABLE 36
CFU Counts

| T(days) | Sample | Dilution | Counts | Average | CFU/mL |
|---|---|---|---|---|---|
| 0 | 50% PPG Lead #3 | 8 | (86, 82, 81, 83, 100) | 86.40 | 8.64E+09 |
| | Bacillus 250X, Tap Water | | | | |
| 63 | 50% PPG Lead #3 | 8 | (60, 51) | 55.50 | 5.55E+09 |
| | Bacillus 250X, Tap Water | | | | |
| 350 | 50% PPG Lead #3 | 8 | (70, 86, 78, 70, 92) | 79.20 | 7.92E+09 |
| | Bacillus 250X, Tap Water | | | | |

TABLE 37
CFU Counts

| T(days) | Sample | Dilution | Counts | Average | CFU/mL |
|---|---|---|---|---|---|
| 0 | Lead #3 Bacillus 1/250X, Tap Water | 6 | (35, 41, 39, 27, 59) | 40.20 | 4.02E+07 |
| 63 | Lead #3 Bacillus 1/250X, Tap Water | 6 | (43, 38, 18, 31) | 32.50 | 3.25E+07 |
| 162 | Lead #3 Bacillus 1/250X, Tap Water | 6 | (21, 16, 9, 13, 22) | 16.20 | 1.62E+07 |
| 170 | Lead #3 Bacillus 1/250X, Tap Water | 6 | (61, 63, 51, 40, 57) | 54.40 | 5.44E+07 |
| 350 | Lead #3 Bacillus 1/250X, Tap Water | 6 | (14, 25, 22, 18, 17) | 19.20 | 1.92E+07 |
| 371 | Lead #3 Bacillus 1/250X, Tap Water | 6 | (9, 14, 5, 13, 11) | 10.40 | 1.04E+07 |

TABLE 38
CFU Counts

| T(days) | Sample | Dilution | Counts | Average | CFU/mL |
|---|---|---|---|---|---|
| 63 | 50% PPG Lead #3 | 8 | (15, 13, 23, 8, 13) | 14.40 | 1.44E+09 |
| | Bacillus 250X, Tap Water 42 C. | | | | |
| 288 | 50% PPG Lead #3 | 8 | (37, 23, 20, 30) | 27.50 | 2.75E+09 |
| | Bacillus 250X, Tap Water 42 C. | | | | |

TABLE 39
CFU Counts

| T(days) | Sample | Dilution | Counts | Average | CFU/mL |
|---|---|---|---|---|---|
| 13 | Lead Bacillus 250X, Tap Water 42 C. | 8 | (167, 239, 180, 273, 185) | 208.80 | 2.09E+10 |
| 69 | Lead Bacillus 250X, Tap Water 42 C. | 8 | (25, 25, 20, 24, 30) | 24.80 | 2.48E+09 |
| 344 | Lead Bacillus 250X, Tap Water 42 C. | 8 | (31, 49, 28, 20) | 32.00 | 3.17E+09 |

TABLE 40
CFU Counts

| T(days) | Sample | Dilution | Counts | Average | CFU/mL |
|---|---|---|---|---|---|
| 13 | Lead Bacillus 250X, DI Water 42 C. | 8 | — | — | — |
| 69 | Lead Bacillus 250X, DI Water 42 C. | 8 | (55, 74, 88) | 72.33 | 7.23E+09 |
| 344 | Lead Bacillus 250X, DI Water 42 C. | 8 | (8, 5, 6, 20, 24) | 12.60 | 1.26E+09 |

TABLE 41
CFU Counts

| T(days) | Sample | Dilution | Counts | Average | CFU/mL |
|---|---|---|---|---|---|
| 0 | Lead Bacillus #4 250x Concentrate, | 8 | (164, 187, 165, 153, 152) | 164.20 | 1.64E+10 |
| | Tap Water | | | | |
| 187 | Lead Bacillus #4 250x Concentrate, | 8 | (108, 96, 100, 117, 94) | 103.00 | 1.03E+10 |
| | Tap Water | | | | |

TABLE 42
CFU Counts

| T(days) | Sample | Dilution | Counts | Average | CFU/mL |
|---|---|---|---|---|---|
| 0 | Lead Bacillus #4 1/250 RTU, Tap Water | 6 | (46, 44, 31, 43, 54) | 43.60 | 4.36E+07 |
| 4 | Lead Bacillus #4 1/250 RTU, Tap Water | 6 | (31, 30, 30, 32, 35) | 31.60 | 3.16E+07 |
| 7 | Lead Bacillus #4 1/250 RTU, Tap Water | 6 | (62, 56, 94, 63, 73) | 69.60 | 6.96E+07 |
| 187 | Lead Bacillus #4 1/250 RTU, Tap Water | 6 | (33, 36, 48, 35, 35) | 37.40 | 3.74E+07 |
| 208 | Lead Bacillus #4 1/250 RTU, Tap Water | 6 | (41, 42, 34, 28, 26) | 34.20 | 3.42E+07 |

TABLE 43
CFU Counts

| T(days) | Sample | Dilution | Counts | Average | CFU/mL |
|---|---|---|---|---|---|
| 0 | Lead Bacillus 40x, Tap Water | 7 | (190, 180, 175, 303) | 212.00 | 2.12E+09 |
| 82 | Lead Bacillus 40x, Tap Water | 7 | (135, 137, 140, 154, 142) | 141.60 | 1.42E+09 |
| 84 | Lead Bacillus 40x, Tap Water | 7 | (213, 237, 192, 231, 220) | 218.60 | 2.19E+09 |

TABLE 44
CFU Counts

| T(days) | Sample | Dilution | Counts | Average | CFU/mL |
|---|---|---|---|---|---|
| 0 | Lead Bacillus 40x RTU, Tap Water | 6 | (49, 23, 26, 35) | 33.25 | 3.33E+07 |
| 4 | Lead Bacillus 40x RTU, Tap Water | 6 | (37, 42, 39, 35, 37) | 38.00 | 3.80E+07 |
| 7 | Lead Bacillus 40x RTU, Tap Water | 6 | (18, 30, 21, 25, 20) | 22.80 | 2.28E+07 |
| 7 | Lead Bacillus 40x RTU, Tap Water | 6 | (47, 55, 50, 55) | 51.75 | 5.18E+07 |
| 82 | Lead Bacillus 40x RTU, Tap Water | 6 | (25, 22, 20, 20, 28) | 23.00 | 2.30E+07 |
| 84 | Lead Bacillus 40x RTU, Tap Water | 6 | (17, 22, 21, 22, 18) | 20.00 | 2.00E+07 |
| 104 | Lead Bacillus 40x RTU, Tap Water | 6 | (18, 16, 19, 15) | 17.00 | 1.70E+07 |
| 183 | Lead Bacillus 40x RTU, Tap Water | 6 | (5, 5, 2, 1, 4) | 3.40 | 3.40E+06 |
| 184 | Lead Bacillus 40x RTU, Tap Water | 6 | (6, 6, 5, 6, 3) | 5.20 | 5.20E+06 |
| 249 | Lead Bacillus 40x RTU, Tap Water | 6 | (1, 6, 2, 1) | 2.50 | 2.50E+06 |

TABLE 45
CFU Counts

| T(days) | Sample | Dilution | Counts | Average | CFU/mL |
|---|---|---|---|---|---|
| 0 | Lead Bacillus 40x RTU, Tap Water T(88) | 6 | (76, 84, 90, 87) | 84.25 | 8.43E+07 |
| 16 | Lead Bacillus 40x RTU, Tap Water T(88) | 6 | (46, 61, 37, 41) | 46.25 | 4.63E+07 |
| 17 | Lead Bacillus 40x RTU, Tap Water T(88) | 6 | (36, 37, 41, 36, 38) | 37.60 | 3.76E+07 |
| 95 | Lead Bacillus 40x RTU, Tap Water T(88) | 6 | (25, 19, 21, 21, 11) | 19.40 | 1.94E+07 |
| 96 | Lead Bacillus 40x RTU, Tap Water T(88) | 6 | (27, 38, 18, 33, 18) | 26.80 | 2.68E+07 |
| 162 | Lead Bacillus 40x RTU, Tap Water T(88) | 6 | (21, 22, 31, 25, 25) | 24.80 | 2.48E+07 |

TABLE 46
CFU Counts

| T(days) | Sample | Dilution | Counts | Average | CFU/mL |
|---|---|---|---|---|---|
| 0 | Lead Bacillus 80x, Tap Water | 8 | (29, 17, 34, 27) | 26.75 | 2.68E+09 |
| 1 | Lead Bacillus 80x, Tap Water | 8 | (84, 82, 74, 83, 81) | 80.80 | 8.08E+09 |
| 4 | Lead Bacillus 80x, Tap Water | 8 | (51, 40, 62, 46, 46) | 49.00 | 4.90E+09 |
| 82 | Lead Bacillus 80x, Tap Water | 8 | (32, 35, 35, 31, 22) | 31.00 | 3.10E+09 |
| 84 | Lead Bacillus 80x, Tap Water | 8 | (43, 48, 41, 45, 41) | 43.60 | 4.36E+09 |

TABLE 47
CFU Counts

| T(days) | Sample | Dilution | Counts | Average | CFU/mL |
|---|---|---|---|---|---|
| 0 | Lead Bacillus 80x RTU, Tap Water | 6 | (66, 64, 60, 81, 74) | 69.00 | 6.90E+07 |
| 4 | Lead Bacillus 80x RTU, Tap Water | 6 | (33, 48, 26, 24, 29) | 32.00 | 3.20E+07 |
| 7 | Lead Bacillus 80x RTU, Tap Water | 6 | (24, 24, 28, 20) | 24.00 | 2.40E+07 |
| 7 | Lead Bacillus 80x RTU, Tap Water | 6 | (41, 50, 66) | 52.33 | 5.23E+07 |
| 82 | Lead Bacillus 80x RTU, Tap Water | 6 | (29, 34, 28) | 30.33 | 3.03E+07 |
| 84 | Lead Bacillus 80x RTU, Tap Water | 6 | (41, 33, 37, 49, 60) | 44.00 | 4.40E+07 |
| 104 | Lead Bacillus 80x RTU, Tap Water | 6 | (13, 19, 11, 19) | 15.50 | 1.55E+07 |
| 105 | Lead Bacillus 80x RTU, Tap Water | 6 | (19, 17, 15, 14, 16) | 16.20 | 1.62E+07 |
| 105 | Lead Bacillus 80x RTU, Tap Water | 6 | (17, 15, 12, 23, 18) | 17.00 | 1.70E+07 |
| 182 | Lead Bacillus 80x RTU, Tap Water | 6 | (6, 6, 13, 1, 4) | 6.00 | 6.00E+06 |
| 183 | Lead Bacillus 80x RTU, Tap Water | 6 | (15, 10, 10, 11, 11) | 11.40 | 1.14E+07 |
| 250 | Lead Bacillus 80x RTU, Tap Water | 6 | (8, 4, 12, 4, 5) | 6.60 | 6.60E+06 |

TABLE 48
CFU Counts

| T(days) | Sample | Dilution | Counts | Average | CFU/mL |
|---|---|---|---|---|---|
| 0 | Lead Bacillus 100x, Tap Water | 8 | (106, 100, 97, 99, 125) | 105.40 | 1.05E+10 |
| 1 | Lead Bacillus 100x, Tap Water | 8 | (48, 49, 57, 54, 36) | 48.80 | 4.88E+09 |
| 82 | Lead Bacillus 100x, Tap Water | 8 | (44, 49, 41, 44) | 44.50 | 4.45E+09 |
| 84 | Lead Bacillus 100x, Tap Water | 8 | (66, 53, 70, 69, 71) | 65.80 | 6.58E+09 |

TABLE 49
CFU Counts

| T(days) | Sample | Dilution | Counts | Average | CFU/mL |
|---|---|---|---|---|---|
| 0 | Lead Bacillus 100x RTU, Tap Water | 6 | (37, 34, 41, 35, 36) | 36.60 | 3.66E+07 |
| 4 | Lead Bacillus 100x RTU, Tap Water | 6 | (13, 20, 21, 27) | 20.25 | 2.03E+07 |
| 6 | Lead Bacillus 100x RTU, Tap Water | 6 | (27, 33, 42, 28, 35) | 33.00 | 3.30E+07 |
| 6 | Lead Bacillus 100x RTU, Tap Water | 6 | (101, 90, 87, 72, 78) | 85.60 | 8.56E+07 |
| 82 | Lead Bacillus 100x RTU, Tap Water | 6 | (23, 43, 29, 25, 42) | 32.40 | 3.24E+07 |
| 84 | Lead Bacillus 100x RTU, Tap Water | 6 | (31, 21, 20, 27, 24) | 24.60 | 2.46E+07 |
| 104 | Lead Bacillus 100x RTU, Tap Water | 6 | (15, 14, 34, 28, 25) | 23.20 | 2.32E+07 |
| 182 | Lead Bacillus 100x RTU, Tap Water | 6 | (6, 10, 11, 20, 12) | 11.80 | 1.18E+07 |
| 183 | Lead Bacillus 100x RTU, Tap Water | 6 | (26, 21, 30, 22) | 24.75 | 2.48E+07 |
| 250 | Lead Bacillus 100x RTU, Tap Water | 6 | (18, 19, 22, 13) | 18.00 | 1.80E+07 |

TABLE 50
CFU Counts

| T(days) | Sample | Dilution | Counts | Average | CFU/mL |
|---|---|---|---|---|---|
| 0 | Direct to RTU Lead Bacillus, | 6 | (57, 36, 38, 50, 38) | 43.80 | 4.38E+07 |
| | 250x Equivalent | | | | |
| 3 | Direct to RTU Lead Bacillus, | 6 | (29, 26, 34, 27, 23) | 27.80 | 2.78E+07 |
| | 250x Equivalent | | | | |
| 6 | Direct to RTU Lead Bacillus, | 6 | (62, 62, 43, 53, 53) | 54.60 | 5.46E+07 |
| | 250x Equivalent | | | | |
| 79 | Direct to RTU Lead Bacillus, | 6 | (22, 39, 27, 27, 28) | 28.60 | 2.86E+07 |
| | 250x Equivalent | | | | |
| 80 | Direct to RTU Lead Bacillus, | 6 | (36, 44, 47, 33, 33) | 38.60 | 3.86E+07 |
| | 250x Equivalent | | | | |
| 147 | Direct to RTU Lead Bacillus, | 6 | (62, 77, 57, 73) | 67.25 | 6.73E+07 |
| | 250x Equivalent | | | | |
| 272 | Direct to RTU Lead Bacillus, | 6 | (158, 180, 48, 38) | 106.00 | 1.06E+08 |
| | 250x Equivalent | | | | |

TABLE 51
CFU Counts

| T(days) | Sample | Dilution | Counts | Average | CFU/mL |
|---|---|---|---|---|---|
| 10 | Lead Bacillus 1/250x, 1 of 6, 42 C. | 6 | (28, 31, 38, 25, 25) | 29.40 | 2.94E+07 |
| 64 | Lead Bacillus 1/250x, 1 of 6, 42 C. | 6 | (3, 14, 19, 7, 13) | 11.20 | 1.12E+07 |
| 130 | Lead Bacillus 1/250x, 1 of 6, 42 C. | 6 | (66, 44, 58, 43) | 52.75 | 5.28E+07 |

TABLE 52
CFU Counts

| T(days) | Sample | Dilution | Counts | Average | CFU/mL |
|---|---|---|---|---|---|
| 8 | Lead Bacillus 1/250x, 2 of 6, refrigerated | 6 | (19, 23, 29, 20) | 22.75 | 2.28E+07 |
| 62 | Lead Bacillus 1/250x, 2 of 6, refrigerated | 6 | (24, 20, 29, 22, 22) | 23.40 | 2.34E+07 |
| 128 | Lead Bacillus 1/250x, 2 of 6, refrigerated | 6 | (19, 35, 39) | 31.00 | 3.10E+07 |

TABLE 53
CFU Counts

| T(days) | Sample | Dilution | Counts | Average | CFU/mL |
|---|---|---|---|---|---|
| 8 | Lead Bacillus 1/250x, 3 of 6, Sideways | 6 | (13, 12, 12, 20, 21) | 15.60 | 1.56E+07 |
| 13 | Lead Bacillus 1/250x, 3 of 6, Sideways | 6 | (29, 28, 26, 31, 29) | 28.60 | 2.86E+07 |

TABLE 54
CFU Counts

| T(days) | Sample | Dilution | Counts | Average | CFU/mL |
|---|---|---|---|---|---|
| 8 | Lead Bacillus 1/250x, 4 of 6, Shaken | 6 | (16, 10, 12, 14, 9) | 12.20 | 1.22E+07 |
| | 250 mL bottle | | | | |
| 13 | Lead Bacillus 1/250x, 4 of 6, Shaken | 6 | (5, 11, 7, 9, 6) | 7.60 | 7.60E+06 |
| | 250 mL bottle | | | | |
| 62 | Lead Bacillus 1/250x, 4 of 6, Shaken | 6 | (16, 15, 14, 16, 14,) | 15.00 | 1.50E+07 |
| | 250 mL bottle | | | | |
| 131 | Lead Bacillus 1/250x, 4 of 6, Shaken | 6 | (7, 3, 12, 5) | 6.75 | 6.75E+06 |
| | 250 mL bottle | | | | |

TABLE 55
CFU Counts

| T(days) | Sample | Dilution | Counts | Average | CFU/mL |
|---|---|---|---|---|---|
| 8 | Lead Bacillus 1/250x, 5 of 6, Shaken | 6 | (4, 6, 3, 5, 6) | 4.80 | 4.80E+06 |
| 13 | Lead Bacillus 1/250x, 5 of 6, Shaken | 5 | (55, 60, 52, 76, 68) | 62.20 | 6.22E+06 |
| 14 | Lead Bacillus 1/250x, 5 of 6, Shaken, | 5 | (77, 80, 87, 80, 85) | 81.80 | 8.18E+06 |
| | 0.1 mL Tween 80 Added | | | | |
| 20 | Lead Bacillus 1/250x, 5 of 6, Shaken, | 6 | (32, 27, 35, 40, 27) | 32.20 | 3.22E+07 |
| | 0.1 mL Tween 80 Added | | | | |

TABLE 56
CFU Counts

| T(days) | Sample | Dilution | Counts | Average | CFU/mL |
|---|---|---|---|---|---|
| 56 | Lead Bacillus 1/250x, 5 of 6, Shaken, | 6 | (35, 39, 34, 25) | 33.25 | 3.33E+07 |
| | 0.1 mL Tween 80 Added | | | | |
| 128 | Lead Bacillus 1/250x, 5 of 6, Shaken, | 6 | (17, 8, 17, 12, 15) | 13.80 | 1.38E+07 |
| | 0.1 mL Tween 80 Added | | | | |

TABLE 57
CFU Counts

| T(days) | Sample | Dilution | Counts | Average | CFU/mL |
|---|---|---|---|---|---|
| 8 | Lead Bacillus 1/250x, 6 of 6, Retain | 6 | (30, 41, 39, 28, 22) | 32.00 | 3.20E+07 |
| 62 | Lead Bacillus 1/250x, 6 of 6, Retain | 6 | (27, 28, 17, 25, 14) | 22.20 | 2.22E+07 |
| 63 | Lead Bacillus 1/250x, 6 of 6, Retain | 6 | (26, 23, 27, 24, 14) | 22.80 | 2.28E+07 |
| 131 | Lead Bacillus 1/250x, 6 of 6, Retain | 6 | (18, 18, 24, 21, 27) | 21.60 | 2.16E+07 |

TABLE 58
CFU Counts

| T(days) | Sample | Dilution | Counts | Average | CFU/mL |
|---|---|---|---|---|---|
| 6 | Lead Bacillus 1/250x 1.5% Tween 80 | 6 | (33, 36, 29, 26, 27) | 30.20 | 3.02E+07 |
| 51 | Lead Bacillus 1/250x 1.5% Tween 80 | 6 | (19, 27, 33, 27, 26) | 26.40 | 2.64E+07 |
| 52 | Lead Bacillus 1/250x 1.5% Tween 80 | 6 | (25, 25, 29, 18, 24) | 24.20 | 2.42E+07 |
| 120 | Lead Bacillus 1/250x 1.5% Tween 80 | 6 | (28, 26, 31, 31) | 29.00 | 2.90E+07 |

TABLE 59
CFU Counts

| T(days) | Sample | Dilution | Counts | Average | CFU/mL |
|---|---|---|---|---|---|
| 6 | Lead Bacillus 1/250x 0.05% Tween 80 | 6 | (15, 14, 15, 14) | 14.50 | 1.45E+07 |

TABLE 60
CFU Counts

| T(days) | Sample | Dilution | Counts | Average | CFU/mL |
|---|---|---|---|---|---|
| 0 | Lead #5 Bacillus 1/250x, 0.1% | 6 | (32, 33, 43, 41, 35) | 36.80 | 3.68E+07 |
| | Tween 80, Tap Water, RTU | | | | |
| 0 | Lead #5 Bacillus 1/250x, 0.1% | 6 | (61, 53, 48, 63) | 56.25 | 5.63E+07 |
| | Tween 80, Tap Water, RTU | | | | |
| 19 | Lead #5 Bacillus 1/250x, 0.1% | 6 | (37, 48, 40) | 41.67 | 4.17E+07 |
| | Tween 80, Tap Water, RTU | | | | |
| 35 | Lead #5 Bacillus 1/250x, 0.1% | 6 | (30, 37, 34, 41) | 35.50 | 3.55E+07 |
| | Tween 80, Tap Water, RTU | | | | |
| 92 | Lead #5 Bacillus 1/250x, 0.1% | 6 | (41, 75, 25, 34) | 43.75 | 4.38E+07 |
| | Tween 80, Tap Water, RTU | | | | |
| 141 | Lead #5 Bacillus 1/250x, 0.1% | 6 | (10, 9, 8, 11, 8) | 9.20 | 9.20E+06 |
| | Tween 80, Tap Water, RTU | | | | |
| 142 | Lead #5 Bacillus 1/250x, 0.1% | 6 | (18, 16, 5, 5, 3) | 9.40 | 9.40E+06 |
| | Tween 80, Tap Water, RTU | | | | |
| 145 | Lead #5 Bacillus 1/250x, 0.1% | 6 | (28, 35, 50, 56) | 42.25 | 4.23E+07 |
| | Tween 80, Tap Water, RTU | | | | |
| 145 | Lead #5 Bacillus 1/250x, 0.1% | 6 | (312, 193, 98, 180, 106) | 177.80 | 1.78E+08 |
| | Tween 80, Tap Water, RTU | | | | |
| 146 | Lead #5 Bacillus 1/250x, 0.1% | 6 | (74, 73, 90, 95, 85) | 83.40 | 8.34E+07 |
| | Tween 80, Tap Water, RTU | | | | |
| 147 | Lead #5 Bacillus 1/250x, 0.1% | 6 | (33, 33, 42, 52) | 40.00 | 4.00E+07 |
| | Tween 80, Tap Water, RTU | | | | |
| 155 | Lead #5 Bacillus 1/250x, 0.1% | 6 | (30, 16) | 23.00 | 2.30E+07 |
| | Tween 80, Tap Water, RTU | | | | |
| 155 | Lead #5 Bacillus 1/250x, 0.1% | 6 | (24, 19, 26, 16, 24) | 21.80 | 2.18E+07 |
| | Tween 80, Tap Water, RTU | | | | |
| 170 | Lead #5 Bacillus 1/250x, 0.1% | 6 | (19, 15, 13, 10, 34) | 18.20 | 1.82E+07 |
| | Tween 80, Tap Water, RTU | | | | |
| 170 | Lead #5 Bacillus 1/250x, 0.1% | 6 | (23, 15, 101, 79) | 54.50 | 5.45E+07 |
| | Tween 80, Tap Water, RTU | | | | |
| 215 | Lead #5 Bacillus 1/250x, 0.1% | 6 | (31) | 31.00 | 3.10E+07 |
| | Tween 80, Tap Water, RTU | | | | |
| 216 | Lead #5 Bacillus 1/250x, 0.1% | 6 | (20, 25, 29, 21, 20) | 23.00 | 2.30E+07 |
| | Tween 80, Tap Water, RTU | | | | |

TABLE 61
CFU Counts

| T(days) | Sample | Dilution | Counts | Average | CFU/mL |
|---|---|---|---|---|---|
| 0 | Lead #5 Bacillus 250x Concentrate, | 8 | (86, 91. 91, 99, 77) | 88.80 | 8.88E+09 |
| | 0.1% Tween 80, Tap Water | | | | |
| 86 | Lead #5 Bacillus 250x Concentrate, | 8 | (70, 93, 95, 87, 86) | 86.20 | 8.62E+09 |
| | 0.1% Tween 80, Tap Water | | | | |
| 141 | Lead #5 Bacillus 250x Concentrate, | 8 | (31, 31, 23, 14, 21) | 24.00 | 2.40E+09 |
| | 0.1% Tween 80, Tap Water | | | | |
| 142 | Lead #5 Bacillus 250x Concentrate, | 8 | (15, 17, 12) | 14.67 | 1.47E+09 |
| | 0.1% Tween 80, Tap Water | | | | |
| 146 | Lead #5 Bacillus 250x Concentrate, | 8 | (101, 64, 17, 32, 64) | 55.60 | 5.56E+09 |
| | 0.1% Tween 80, Tap Water | | | | |
| 147 | Lead #5 Bacillus 250x Concentrate, | 8 | (80, 109, 96, 98, 97) | 96.00 | 9.60E+09 |
| | 0.1% Tween 80, Tap Water | | | | |
| 156 | Lead #5 Bacillus 250x Concentrate, | 8 | (96, 81, 78, 94, 85) | 86.80 | 8.68E+09 |
| | 0.1% Tween 80, Tap Water | | | | |

TABLE 62
CFU Counts

| T(days) | Sample | Dilution | Counts | Average | CFU/mL |
|---|---|---|---|---|---|
| 0 | Lead #2 Bacillus 40x Concentrate, | 7 | (220, 219, 227, 219, 184) | 213.80 | 2.14E+09 |
| | Tap Water, 0.02% Tween 80 | | | | |
| 64 | Lead #2 Bacillus 40x Concentrate, | 7 | (177, 198, 186, 170, 153) | 176.80 | 1.77E+09 |
| | Tap Water, 0.02% Tween 80 | | | | |

TABLE 63
CFU Counts

| T(days) | Sample | Dilution | Counts | Average | CFU/mL |
|---|---|---|---|---|---|
| 0 | Lead #2 Bacillus 1/40x RTU | 6 | (44, 48, 50, 72, 41) | 51.00 | 5.10E+07 |
| 64 | Lead #2 Bacillus 1/40x RTU | 6 | (51, 53, 47, 49, 41) | 48.20 | 4.82E+07 |

TABLE 64
CFU Counts

| T(days) | Sample | Dilution | Counts | Average | CFU/mL |
|---|---|---|---|---|---|
| 0 | Lead #2 Bacillus 80x Concentrate, | 8 | (153, 47, 41, 29) | 67.50 | 6.75E+09 |
| | Tap Water, 0.04% Tween 80 | | | | |
| 64 | Lead #2 Bacillus 80x Concentrate, | 8 | (52, 54, 41, 28, 44) | 43.80 | 4.38E+09 |
| | Tap Water, 0.04% Tween 80 | | | | |

TABLE 65
CFU Counts

| T(days) | Sample | Dilution | Counts | Average | CFU/mL |
|---|---|---|---|---|---|
| 0 | Lead #2 Bacillus 1/80x RTU | 6 | (58, 64, 52, 58, 53) | 57.00 | 5.70E+07 |
| 64 | Lead #2 Bacillus 1/80x RTU | 6 | (49, 46, 41, 45, 44) | 45.00 | 4.50E+07 |

TABLE 66
CFU Counts

| T(days) | Sample | Dilution | Counts | Average | CFU/mL |
|---|---|---|---|---|---|
| 0 | Lead #2 Bacillus 100x Concentrate, | 8 | (68, 13, 52, 58, 47) | 47.60 | 4.76E+09 |
| | Tap Water, 0.05% Tween 80 | | | | |
| 65 | Lead #2 Bacillus 100x Concentrate, | 8 | (42, 38, 49, 57, 56) | 48.40 | 4.84E+09 |
| | Tap Water, 0.05% Tween 80 | | | | |

TABLE 67
CFU Counts

| T(days) | Sample | Dilution | Counts | Average | CFU/mL |
|---|---|---|---|---|---|
| 0 | Lead #2 Bacillus 1/100x RTU | 6 | (60, 45, 71, 55, 58) | 57.80 | 5.78E+07 |
| 64 | Lead #2 Bacillus 1/100x RTU | 6 | (44, 45, 53, 58, 35) | 47.00 | 4.70E+07 |

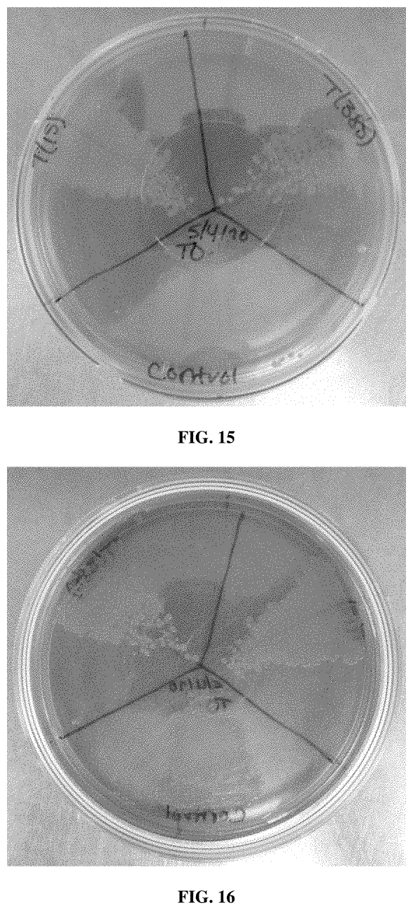

Formulations of this example were also used in Gram negative bacterial and yeast challenges, and representative challenge results are provided by FIGS. 15-20 and Tables 68-71 below. These results clearly establish that compositions according to the present invention had substantially reduced yeast and bacterial contamination relative to a control composition that did not include a Prep B preservative composition according to the present invention.

More specifically, challenge results for a 1/250× Bacillus composition are provided below in Table 68 and FIGS. 15 and 16. Table 68 is at TO, and therefore acts as a control for results at later time intervals.

TABLE 68
Gram Negative Bacterial Challenge Results

| T(days) | Sample | Result |
|---|---|---|
| 0 | Control | + |
| 0 | Lead Bacillus 1/250X T(stock + 15) | + |
| 0 | Lead Bacillus 1/250X T(stock + 385) | +/− |

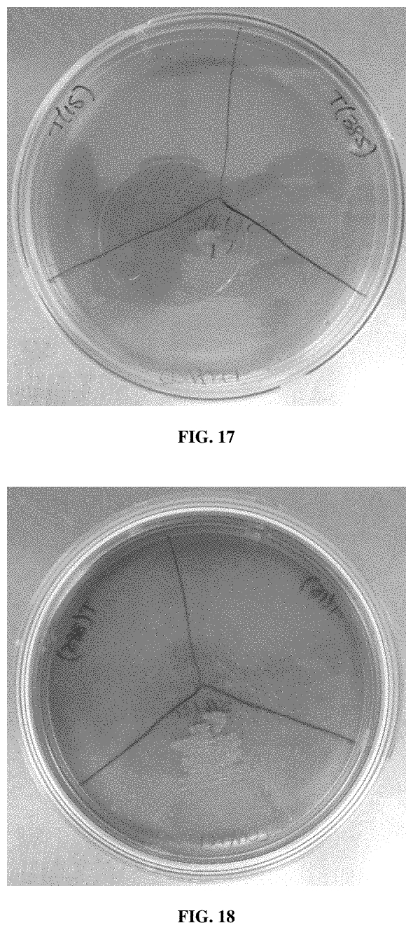

Challenge results for a 1/250× Bacillus composition are provided below in Table 69 and FIGS. 17 and 18. These results establish that both the 250× and RTU retain good long-term preservation integrity. The 250× was produced over a year before this challenge was completed—15 days later an RTU was produced from it (T+15). The T+15 RTU was stored for 1 year and challenged, resulting in an absence of G−. This indicates the RTU's have good long-term preservation properties, as this particular example had 1 year to potentially degrade. 385 days after preparing the 250×, another “fresh” RTU was prepared and challenged (T+385). This was also negative, indicating that the preservation system for the concentrate remained efficacious at least over 385 days.

TABLE 69
Gram Negative Bacterial Challenge Results

| T(days) | Sample | Result |
|---|---|---|
| 2 | Control | + |
| 2 | Lead Bacillus 1/250X T(stock + 15) | − |
| 2 | Lead Bacillus 1/250X T(stock + 385) | − |

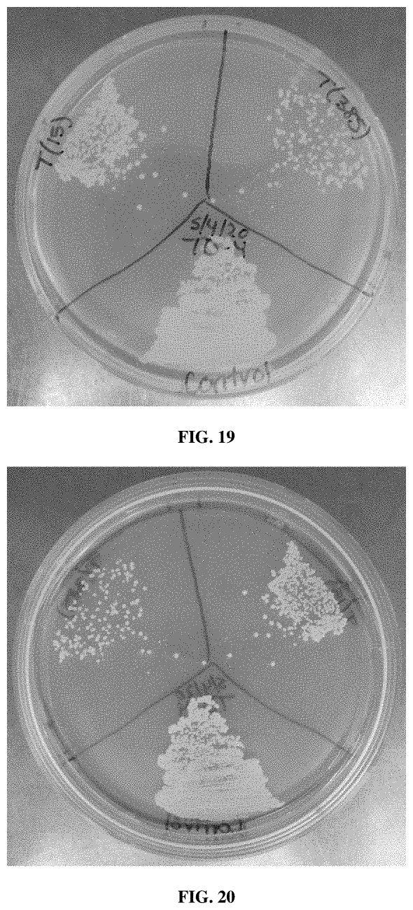

Yeast challenge results for a 1/250× Bacillus composition are provided below in Table 70 and FIGS. 19 and 20 at T0, which acts as a control for results at later test dates.

TABLE 70
Yeast Challenge Results

| T(days) | Sample | Result |
|---|---|---|
| 0 | Control | + |
| 0 | Lead Bacillus 1/250X T(stock + 15) | +/− |
| 0 | Lead Bacillus 1/250X T(stock + 385) | −/+ |

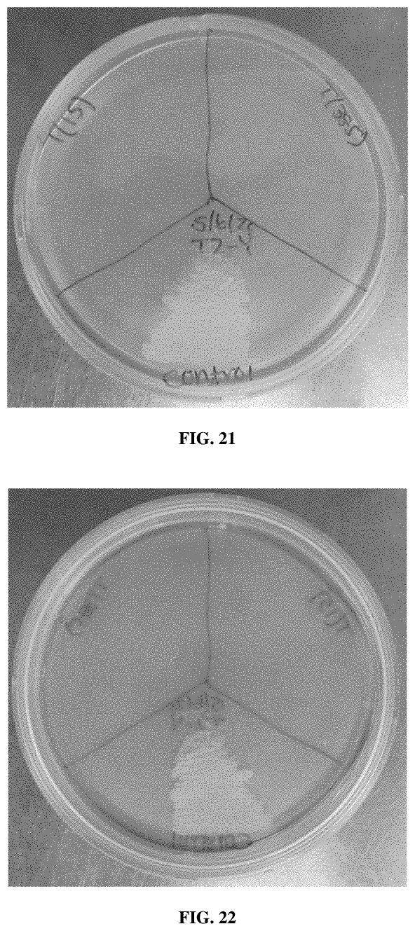

Yeast challenge results for a 1/250× Bacillus composition are provided below in Table 71 and FIGS. 21 and 22. These results establish that the representative tested composition according to the present invention had substantially no contamination, whereas the control was contaminated with yeast at T₂, i.e., only 2 days subsequent to challenge.

TABLE 71
Yeast Challenge Results

| T(days) | Sample | Result |
|---|---|---|
| 2 | Control | + |
| 2 | Lead Bacillus 1/250X T(stock + 15) | − |
| 2 | Lead Bacillus 1/250X T(stock + 385) | − |

In summary, an array of bacterial formulations have been tested. The RTU of the 250× by and large had a stronger/faster effect than any other freshly prepared RTU's tested. More importantly however, was the long-term retention of preservative effect in all its forms (i.e. either fresh RTU's produced from old 250× stock, or simply old RTU's) compared to existing products. Existing formulations tend to lose preservation and suffer from spore die-off in both the concentrate and RTU, often resulting in a low-count and contaminated RTU. This is especially true with the higher CFU/mL formulations, highlighted by the fact that the highest standalone concentrate that can be both definitively preserved and die-off prevented without using disclosed embodiments of the present invention is a 5× composition.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A concentrated microbial composition concentrated by greater than 5 times to at least 250 times a concentration intended for an end-use application, comprising:
a microbial suspension comprising one or more spore forming microbes; and
a preservative system comprising plural different preservatives.

2. The composition according to claim 1, wherein the microbe is a Bacillus selected from Bacillus coagulans, Bacillus pumilus, Bacillus thuringiensis, Bacillus inaquosorum, Bacillus mojavensis, Bacillus megaterium, Bacillus globigii, Bacillus subtilis, Bacillus simplex, Bacillus licheniformis and/or Bacillus amyloliquefaciens.

3. The composition according to claim 1, comprising:
a concentrated bacterial suspension comprising bacterial cells or spores;
a solvent; and
plural preservatives.

4. The composition according to claim 3, further comprising at least one additional component selected from a buffering system, a non-ionic surfactant, Tween 80, odor mitigation components, deodorizers, fragrances, opacifiers, enzymes, such as a cellulase or lipase, proteins, gelling agents, additional preservatives, sanitizing agents, emulsifying agents, API's, oils such as triglycerides, antioxidants, oxidizers, petroleum products, biocides, solvents, and any and all combinations thereof.

5. The composition according to claim 1, comprising 10% to 60% by weight humectant solvent, where the humectant solvent is an alcohol, a diol, a triol, an alkylene glycol, glycerol or a combination thereof.

6. The composition according to claim 1, comprising a paraben preservative, an isothiazolinone preservative, a quaternary ammonium preservative, or a combination thereof.

7. The composition according to claim 6, wherein the quaternary ammonium preservative has a formula R¹R²R³R⁴N⁺X⁻, where R¹-R⁴ are independently selected from H, C₁-C₂₀ alkyl, and aryl.

8. The composition according to claim 7, wherein:
the quaternary ammonium preservative is selected from benzalkonium chloride, cetyl pyridinium chloride, octylphenoxyethoxyethyl dimethyl benzyl ammonium chloride, and combinations thereof;
the paraben preservative is selected from methylparaben and propylparaben; and
the isothiazolinone preservative is selected from 1,2-benzoisothiazolin-3-one (BIT), methylisothiazolinone (MIT), methylchloroisothiazolinone (CMIT), or combinations thereof.

9. The composition according to claim 6, comprising benzalkonium chloride, 1,2-benzoisothiazolin-3-one, and propylparaben.

10. The composition according to claim 1, wherein the composition is formulated using a Prep A composition comprising the microbial suspension and a Prep B composition comprising the preservative system.

11. The composition according to claim 10 wherein the Prep B composition for a 250× formulation comprises:
28% to 29% Proxel GXL comprising 20% benzisothiazolinone;
14% to 15% Stepenquat 65 NF comprising 50% n-alkyldimethylbenzylammonium chloride; and
14% to 15% propylparaben.

12. The composition according to claim 10 wherein the Prep B composition consists essentially of:
28% to 30% Proxel GXL;
14%-15% Stepenquat 65 NF;
14% to 15% propylparaben; and
42% to 43% propylene glycol.

13. The composition according to claim 10, wherein the microbial suspension comprises:
at least one Bacillus species;
a buffer system;
a non-aqueous solvent; and
water.

14. The composition according to claim 1, comprising a substantially decreased bacterial, fungal or yeast contamination relative to a similarly formulated microbial suspension that does not include the preservative system.

15. A ready-to-use composition according to claim 1, comprising:
5 to 7×10⁷ CFU/mL bacterial cells and/or spores;
0.1% to 0.5% propylene glycol;
0.02% propylparaben;
0.04% benzoisothiazolone;
0.02% n-alkyl dimethyl benzyl ammonium chloride;
0.010% dipotassium phosphate;
0.006% monopotassium phosphate;
optionally 0.0004% Tween 80; and
water to desired volume.

16. A 100× concentrate composition according to claim 1, comprising:
5 to 7×10⁹ CFU/mL bacterial Cells and/or spores;
36% propylene glycol;
2% propylparaben;
4% benzoisothiazolone;
2% n-alkyl dimethyl benzyl ammonium chloride;
1% dipotassium phosphate;
0.6% monopotassium phosphate;
optionally 0.1% Tween 80; and
water to volume.

17. A 250× concentrate composition according to claim 1, comprising:
1 to 2×10¹⁰ CFU/mL bacterial Cells and/or spores;
41% propylene glycol:
5% propylparaben;
2% benzoisothiazolone;
2.5% n-alkyl dimethyl benzyl ammonium chloride;
2.5% dipotassium phosphate;
1.5% monopotassium phosphate;
optionally 0.1% Tween 80; and
water to volume.

18. A preservative system, comprising:
an isothiazolinone preservative;
an ammonium halide preservative;
a paraben preservative; and
an alkylene glycol.

19. The preservative system according to claim 18, comprising:
28%-29% of a 15%-20% aqueous dipropylene glycol solution comprising 1,2-benzoisothiazolin-3-one;
5%-7% of a 4%-5% ethyl alcohol composition comprising 47%-53% C₈₋₁₈ n-alkyl dimethyl benzyl ammonium chloride;
5%-7% propylparaben; and
42%-45% propylene glycol.

20. A combination, comprising:
a microbial suspension comprising a phosphate salt or combination of phosphate salts, an alkylene glycol, and water, wherein the microbial suspension comprises a Bacillus selected from Bacillus coagulans, Bacillus pumilus, Bacillus thuringiensis, Bacillus inaquosorum, Bacillus mojavensis, Bacillus megaterium, Bacillus globigii, Bacillus subtilis, Bacillus simplex, Bacillus licheniformis and/or Bacillus amyloliquefaciens; and
a preservative system comprising (i) a humectant solvent selected from an alcohol, a polyol, an alkylene glycol, glycerol, or a combination thereof, and (ii) plural preservatives selected from a paraben preservative, an isothiazolinone preservative, a quaternary ammonium preservative, and combinations thereof.

21. A method, comprising:
providing or obtaining a concentrated, stabilized and preserved microbial suspension composition according to claim 1;
diluting the concentrated, stabilized and preserved microbial suspension composition with water, an alkylene glycol or a combination thereof to form a diluted microbial suspension composition; and
using the diluted microbial suspension composition.

22. The method according to claim 21 comprising using the diluted microbial suspension composition as a cleaning product, as an odor control aid, to drain lines, as a janitorial product, as an agricultural product, as a product to treat wastewater, as an environmental product, or as a processing aid.

23. A method for making a concentrated, stabilized and preserved microbial suspension composition, comprising combining a microbial suspension with a Prep B preservative system.
